(19) United States
(12) Patent Application Publication
(10) Pub. No.: US 2022/0241374 A1
(43) Pub. Date: Aug. 4, 2022

(54) TREATMENT OF NON-ALCOHOLIC STEATOHEPATITIS

(71) Applicant: INMUNE BIO INC., Boca Raton, FL (US)

(72) Inventor: RAYMOND J. TESI, Martha's Vineyard, MA (US)

(73) Assignee: INMUNE BIO INC., Boca Raton, FL (US)

(21) Appl. No.: 17/611,487

(22) PCT Filed: May 13, 2020
(86) PCT No.: PCT/US20/32649

§ 371 Date: Nov. 15, 2021

Related U.S. Application Data
(60) Provisional application No. 62/847,171, filed on May 13, 2019.

(51) Int. Cl. A61K 38/19; A61P 1/16; A61K 9/00; G01N 33/68

(52) U.S. Cl. CPC A61K 38/191; G01N 33/6893; A61K 9/0019; A61P 1/16

(57) ABSTRACT

A method of treating non-alcoholic steatohepatitis (NASH) in a subject includes administering to the subject a therapeutically effective amount of a selective inhibitor of solTNF-α, whereby the subject is treated. In some embodiments, the selective inhibitor of solTNF-α includes a DN-TNF-α protein and/or a nucleic acid encoding the DN-TNF-α protein. In some embodiments, the DN-TNF-α protein includes XPRO1595.

### TECHNICAL FIELD

The invention is directed to a method for treating non-alcoholic steatohepatitis (NASH) in a subject.

More particularly, the invention is directed to a method for treating such a subject suffering from NASH by administering a selective inhibitor of soluble tumor necrosis factor alpha (solTNF-α), and more specifically, wherein the selective inhibitor of solTNF-αincludes a dominant negative tumor necrosis alpha (DN-TNF-α) protein or a nucleic acid encoding the DN-TNF-α protein.

### BACKGROUND ART

Non-alcoholic fatty liver disease (NAFLD) is an emerging global health problem and a potential risk factor for type 2 diabetes, cardiovascular disease, and chronic kidney disease. Nonalcoholic steatohepatitis (NASH), an advanced form of NAFLD, is a predisposing factor for development of cirrhosis and hepatocellular carcinoma. The increasing prevalence of NASH emphasizes the need for novel therapeutic approaches. Therapeutic drugs against NASH are not yet available. The pathogenesis of NASH involves multiple intracellular/extracellular events in various cell types in the liver or crosstalk events between the liver and other organs. A review of the current findings and knowledge regarding the pathogenesis of NASH is detailed by Kim K H et al. (“Pathogenesis of nonalcoholic steatohepatitis and hormone-based therapeutic approaches” Front Endocrinol 2018; 9: 485).

The diagnosis of NASH is established by the presence of a characteristic pattern of steatosis, inflammation and hepatocellular ballooning on liver biopsies in the absence of significant alcohol consumption. A scoring system for NAFLD, the “NAFLD activity score (NAS)”, was developed and validated by the NIDDK sponsored Nonalcoholic Steatohepatitis Clinical Research Network (NASH CRN) Pathology Committee (Kleiner, D E et al., Design and validation of a histological scoring system for nonalcoholic fatty liver disease. Hepatology. 2005; 41(6):1313-1321). NAS is the unweighted sum of steatosis, lobular inflammation, and hepatocellular ballooning scores.

Another review of the current state of the art concerning diagnosis and treatment of NAFLD/NASH is described by Leoni, Simona et al. (“Current guidelines for the management of non-alcoholic fatty liver disease: A systematic review with comparative analysis.” World journal of gastroenterology vol. 24, 30 (2018): 3361-3373).

To assist screening and evaluation of drug candidates for nonalcoholic steatohepatitis, a murine model (“STAM Model”) was developed and disclosed by M. Fujii, et al. (“A murine model for non-alcoholic steatohepatitis showing evidence of association between diabetes and hepatocellular carcinoma”, Med. Mol. Morphol., 46 (2013), pp. 141-152).

At the time of this disclosure, NAS is one of the clinical endpoints for assessing the activity of NASH (Sanyal A J. et al., Hepatology, 2011; 54:344), and thus is the key preclinical endpoint in clinical translation.

### SUMMARY OF INVENTION

### Technical Problem

Lifestyle interventions, such as dietary caloric restriction and exercise, currently the cornerstone of therapy for NAFLD/NASH, can be difficult to achieve and maintain, underscoring the dire need for pharmacotherapy.

### Solution to Problem

It was surprisingly discovered that administration of a selective inhibitor of solTNFα showed significant decreases in NAS and fibrosis area compared with a vehicle group in a preclinical murine model (STAM Model). In particular, the selective inhibitor of solTNFα was a DN-TNF-α protein, and more particularly, XPRO1595 (recently becoming known to those with skill in the art as “INB03”).

In the STAM Model experiments, a test group (treated with the selective inhibitor of solTNFα) showed light to moderate improvements in steatosis and lobular inflammation compared with the vehicle group, and, perhaps more surprisingly, the test group showed no hepatocyte ballooning at all, which was significantly less than the vehicle group. Accordingly, the test group resulted in a significantly lower NAS. Moreover, the test group showed a significant reduction in fibrosis area (Sirius red-positive area) compared with the vehicle group. These results indicate that treatment of NASH may be accomplished with the administration of a therapeutically effective amount of a selective inhibitor of solTNF-α, more specifically, a DN-TNF-α protein, and still more particularly, XPRO1595.

Accordingly, the solution to the problem includes, inter alia: administering, to a subject diagnosed with NAFLD and/or NASH, a therapeutically effective amount of a selective inhibitor of solTNF-α, such as a DN-TNF-α protein or a nucleic acid encoding the DN-TNF-α protein, including the compound known as XPRO1595.

Other features and aspects concerning the invention and/or solutions to the aforementioned problem will be recognized by one having skill in the art upon a thorough review of the appended details and descriptions, in particular when reviewed in conjunction with the enclosed drawings.

### Advantageous Effects of Invention

The method described herein, namely, administering to a subject diagnosed with NAFLD and/or NASH a therapeutically effective amount of a selective inhibitor of solTNF-α, has been shown to lower NAS and reduce fibrosis area in a preclinical STAM Model, and thus provides a reasonable basis to conclude that the method may be useful for application in a human subject, with proper regulatory approval and subject to validation in clinical trials.

### BRIEF DESCRIPTION OF DRAWINGS

FIG. 1A shows the nucleic acid sequence of human TNF-α (SEQ ID NO:1). An additional six histidine codons, located between the start codon and the first amino acid, are underlined.

FIG. 1B shows the amino acid sequence of human TNF-α (SEQ ID NO:2) with an additional 6 histidines (underlined) between the start codon and the first amino acid. Amino acids changed in exemplary TNF-α variants are shown in bold.

FIG. 1C shows the amino acid sequence of human TNF-α (SEQ ID NO:3).

FIG. 2 shows the positions and amino acid changes in certain TNF-α variants.

FIG. 3 shows changes in subject body weight throughout the experiment of Example 1.

FIG. 4 shows subject body weight measured from murine subjects.

FIG. 5A shows subject liver weight measured from murine subjects.

FIG. 5B shows liver-to-body weight ratio measured from murine subjects.

FIG. 6A shows plasma ALT measured from murine subjects.

FIG. 6B shows liver triglyceride measured from murine subjects.

FIG. 7A shows a representative photomicrograph of HE-stained liver section for Vehicle subject at 50× magnification.

FIG. 7B shows a representative photomicrograph of HE-stained liver section for Vehicle subject at 200× magnification.

FIG. 7C shows a representative photomicrograph of HE-stained liver section for Compound subject at 50× magnification.

FIG. 7D shows a representative photomicrograph of HE-stained liver section for Compound subject at 200× magnification.

FIG. 7E shows NAFLD Activity Score (NAS) for the murine cohort.

FIG. 7F shows steatosis score for the murine cohort.

FIG. 7G shows inflammation score for the murine cohort.

FIG. 7H shows ballooning score for the murine cohort.

FIG. 8A shows a representative photomicrograph of Sirius red-stained liver section for the Vehicle group.

FIG. 8B shows a representative photomicrograph of Sirius red-stained liver section for the Compound group.

FIG. 8C shows fibrosis area (Sirius red-positive area) for the Compound group compared with the Vehicle group.

FIG. 9A shows relative TNF-α mRNA expression for the Compound group compared with the Vehicle group.

FIG. 9B shows relative INF-γ mRNA expression for the Compound group compared with the Vehicle group.

FIG. 9C shows relative Collagen Type 1 mRNA expression for the Compound group compared with the Vehicle group.

FIG. 9D shows relative TGF-β mRNA expression for the Compound group compared with the Vehicle group.

FIG. 9E shows relative TIMP-1 mRNA expression for the Compound group compared with the Vehicle group.

FIG. 9F shows relative MCP-1 mRNA expression for the Compound group compared with the Vehicle group.

FIG. 10A shows a representative photomicrograph of F4/80-immunostained liver section for a Vehicle group from the experiment associated with Example 2.

FIG. 10B shows a representative photomicrograph of F4/80-immunostained liver section for a Compound group from the experiment associated with Example 2.

FIG. 10C shows inflammation area for each of the Vehicle and Compound groups from the experiment associated with Example 2.

FIG. 11 depicts a method for treating a subject diagnosed with or suffering from non-alcoholic steatohepatitis (NASH).

### DESCRIPTION OF EMBODIMENTS

Disclosed herein is the novel and unexpected finding that a selective inhibition of soluble TNF-α lowers the NAFLD Activity Score (NAS) and reduces fibrosis area in a subject diagnosed with non-alcoholic steatohepatitis (NASH), an advanced form of non-alcoholic fatty liver disease (NAFLD). A method which applies this unexpected finding is disclosed, and comprises the step of: administering, to a subject diagnosed with NAFLD and/or NASH, a therapeutically effective amount of a selective inhibitor of solTNF-α, such as a DN-TNF-α protein or a nucleic acid encoding the DN-TNF-α protein, for example, the DN-TNF-α protein known as XPRO1595.

### Selective Inhibitors of Soluble Tumor Necrosis Factor

Proteins with TNF-α antagonist activity, and nucleic acids encoding these proteins, were previously discovered which function to inhibit the soluble form of TNF-α (solTNF-α) without inhibiting transmembrane TNF-α (tmTNF-α); collectively these proteins and nucleic acids encoding these proteins are herein collectively referred to as “selective inhibitors of solTNF-α”.

Examples of selective inhibitors of solTNF-α are disclosed in U.S. Pat. Nos. 7,056,695; 7,101,974; 7,144,987; 7,244,823; 7,446,174; 7,662,367; and 7,687,461; the entire contents of each of which is hereby incorporated by reference.

Preferred selective inhibitors of solTNF-α may be dominant negative TNF-α proteins, referred to herein as “DNTNF-α,” “DN-TNF-α proteins,” “TNFα variants,” “TNFα variant proteins,” “variant TNF-α,” “variant TNF-α,” and the like. By “variant TNF-α” or “TNF-α proteins” is meant TNFα or TNF-α proteins that differ from the corresponding wild type protein by at least 1 amino acid. Thus, a variant of human TNF-α is compared to SEQ ID NO:1 (nucleic acid including codons for 6 histidines), SEQ ID NO:2 (amino acid including 6 N-terminal histidines) or SEQ ID NO:3 (amino acid without 6 N-terminal histidines). DN-TNF-α proteins are disclosed in detail in U.S. Pat. No. 7,446,174, which is incorporated herein in its entirety by reference. As used herein variant TNF-α or TNF-α proteins include TNF-α monomers, dimers or trimers. Included within the definition of “variant TNF-α” are competitive inhibitor TNF-α variants. While certain variants as described herein, one of skill in the art will understand that other variants may be made while retaining the function of inhibiting soluble but not transmembrane TNF-α.

Thus, the proteins useful in various aspects of the invention are antagonists of wild type TNF-α. By “antagonists of wild type TNF-α” is meant that the variant TNF-α protein inhibits or significantly decreases at least one biological activity of wild-type TNF-α.

In a preferred embodiment the variant is antagonist of soluble TNF-α, but does not significantly antagonize transmembrane TNF-α, e.g., DN-TNF-α protein as disclosed herein inhibits signaling by soluble TNF-α, but not transmembrane TNF-α. By “inhibits the activity of TNF-α” and grammatical equivalents is meant at least a 10% reduction in wild-type, soluble TNF-α, more preferably at least a 50% reduction in wild-type, soluble TNF-α activity, and even more preferably, at least 90% reduction in wild-type, soluble TNF-α activity. Preferably there is an inhibition in wild-type soluble TNF-α activity in the absence of reduced signaling by transmembrane TNF-α. In a preferred embodiment, the activity of soluble TNF-α is inhibited while the activity of transmembrane TNF-α is substantially and preferably completely maintained.

The TNF proteins useful in various embodiments of the invention have modulated activity as compared to wild type proteins. In a preferred embodiment, variant TNF-α proteins exhibit decreased biological activity (e.g. antagonism) as compared to wild type TNF-α, including but not limited to, decreased binding to a receptor (p55, p75 or both), decreased activation and/or ultimately a loss of cytotoxic activity. By “cytotoxic activity” herein refers to the ability of a TNF-α variant to selectively kill or inhibit cells. Variant TNF-α proteins that exhibit less than 50% biological activity as compared to wild type are preferred. More preferred are variant TNF-α proteins that exhibit less than 25%, even more preferred are variant proteins that exhibit less than 15%, and most preferred are variant TNF-α proteins that exhibit less than 10% of a biological activity of wild-type TNF-α. Suitable assays include, but are not limited to, caspase assays, TNF-α cytotoxicity assays, DNA binding assays, transcription assays (using reporter constructs), size exclusion chromatography assays and radiolabeling/immuno-precipitation,), and stability assays (including the use of circular dichroism (CD) assays and equilibrium studies), according to methods know in the art.

In one embodiment, at least one property critical for binding affinity of the variant TNF-α proteins is altered when compared to the same property of wild type TNF-α and in particular, variant TNF-α proteins with altered receptor affinity are preferred. Particularly preferred are variant TNF-α with altered affinity toward oligomerization to wild type TNF-α. Thus, the invention makes use of variant TNF-α proteins with altered binding affinities such that the variant TNF-α proteins will preferentially oligomerize with wild type TNF-α, but do not substantially interact with wild type TNF receptors, i.e., p55, p75. “Preferentially” in this case means that given equal amounts of variant TNF-α monomers and wild type TNF-α monomers, at least 25% of the resulting trimers are mixed trimers of variant and wild type TNF-α, with at least about 50% being preferred, and at least about 80-90% being particularly preferred. In other words, it is preferable that the variant TNF-α proteins implemented in embodiments of the invention have greater affinity for wild type TNF-α protein as compared to wild type TNF-α proteins. By “do not substantially interact with TNF receptors” is meant that the variant TNF-α proteins will not be able to associate with either the p55 or p75 receptors to significantly activate the receptor and initiate the TNF signaling pathway(s). In a preferred embodiment, at least a 50% decrease in receptor activation is seen, with greater than 50%, 75%, 80-90% being preferred.

In some embodiments, the variants of the invention are antagonists of both soluble and transmembrane TNF-α. However, as described herein, preferred variant TNF-α proteins are antagonists of the activity of soluble TNF-α but do not substantially affect the activity of transmembrane TNF-α. Thus, a reduction of activity of the heterotrimers for soluble TNF-α is as outlined above, with reductions in biological activity of at least 10%, 25, 50, 75, 80, 90, 95, 99 or 100% all being preferred. However, some of the variants outlined herein comprise selective inhibition; that is, they inhibit soluble TNF-α activity but do not substantially inhibit transmembrane TNF-α. In these embodiments, it is preferred that at least 80%, 85, 90, 95, 98, 99 or 100% of the transmembrane TNF-α activity is maintained. This may also be expressed as a ratio; that is, selective inhibition can include a ratio of inhibition of soluble to transmembrane TNF-α. For example, variants that result in at least a 10:1 selective inhibition of soluble to transmembrane TNF-α activity are preferred, with 50:1, 100:1, 200:1, 500:1, 1000:1 or higher find particular use in the invention. Thus, one embodiment utilizes variants, such as double mutants at positions 87/145 as outlined herein, that substantially inhibit or eliminate soluble TNF-α activity (for example by exchanging with homotrimeric wild-type to form heterotrimers that do not bind to TNF-α receptors or that bind but do not activate receptor signaling) but do not significantly affect (and preferably do not alter at all) transmembrane TNF-α activity. Without being bound by theory, the variants exhibiting such differential inhibition allow the decrease of inflammation without a corresponding loss in immune response.

In one embodiment, the affected biological activity of the variants is the activation of receptor signaling by wild type TNF-α proteins. In a preferred embodiment, the variant TNF-α protein interacts with the wild type TNF-α protein such that the complex comprising the variant TNF-α and wild type TNF-α has reduced capacity to activate (as outlined above for “substantial inhibition”), and in preferred embodiments is incapable of activating, one or both of the TNF receptors, i.e. p55 TNF-R or p75 TNF-R. In a preferred embodiment, the variant TNF-α protein is a variant TNF-α protein that functions as an antagonist of wild type TNF-α. Preferably, the variant TNF-α protein preferentially interacts with wild type TNF-α to form mixed trimers with the wild type protein such that receptor binding does not significantly occur and/or TNF-α signaling is not initiated. By mixed trimers is meant that monomers of wild type and variant TNF-α proteins interact to form heterotrimeric TNF-α. Mixed trimers may comprise 1 variant TNF-α protein:2 wild type TNF-α proteins, 2 variant TNF-α proteins:1 wild type TNF-α protein. In some embodiments, trimers may be formed comprising only variant TNF-α proteins.

The variant TNF-α antagonist proteins implemented in embodiments of the invention are highly specific for TNF-α antagonism relative to TNF-beta antagonism. Additional characteristics include improved stability, pharmacokinetics, and high affinity for wild type TNF-α. Variants with higher affinity toward wild type TNF-α may be generated from variants exhibiting TNF-α antagonism as outlined above.

Similarly, variant TNF-α proteins, for example are experimentally tested and validated in in vivo and in in vitro assays. Suitable assays include, but are not limited to, activity assays and binding assays. For example, TNF-α activity assays, such as detecting apoptosis via caspase activity can be used to screen for TNF-α variants that are antagonists of wild type TNF-α. Other assays include using the Sytox green nucleic acid stain to detect TNF-induced cell permeability in an Actinomycin-D sensitized cell line. As this stain is excluded from live cells, but penetrates dying cells, this assay also can be used to detect TNF-α variants that are agonists of wild-type TNF-α. By “agonists of wild type TNF-α” is meant that the variant TNF-α protein enhances the activation of receptor signaling by wild type TNF-α proteins. Generally, variant TNF-α proteins that function as agonists of wild type TNF-α are not preferred. However, in some embodiments, variant TNF-α proteins that function as agonists of wild type TNF-α protein are preferred. An example of an NF kappaB assay is presented in Example 7 of U.S. Pat. No. 7,446,174, which is expressly incorporated herein by reference.

In a preferred embodiment, binding affinities of variant TNF-α proteins as compared to wild type TNF-α proteins for naturally occurring TNF-α and TNF receptor proteins such as p55 and p75 are determined. Suitable assays include, but are not limited to, e.g., quantitative comparisons comparing kinetic and equilibrium binding constants, as are known in the art. Examples of binding assays are described in Example 6 of U.S. Pat. No. 7,446,174, which is expressly incorporated herein by reference.

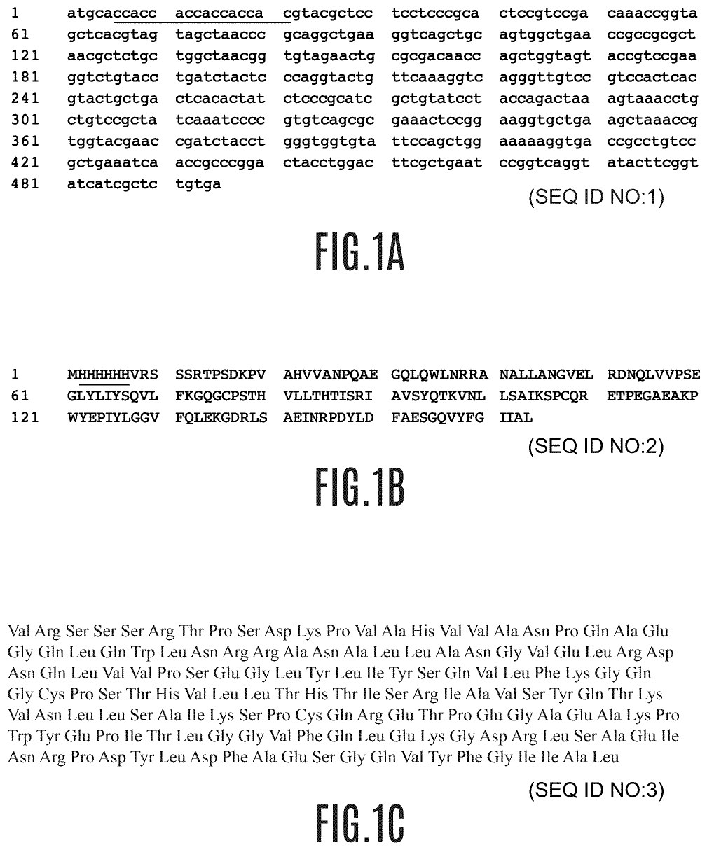

In a preferred embodiment, the variant TNF-α protein has an amino acid sequence that differs from a wild type TNF-α sequence by at least 1 amino acid, with from 1, 2, 3, 4, 5, 6, 7, 8, 9 and 10 amino acids all contemplated, or higher. Expressed as a percentage, the variant TNF-α proteins of the invention preferably are greater than 90% identical to wild-type, with greater than 95, 97, 98 and 99% all being contemplated. Stated differently, based on the human TNF-α sequence of FIG. 1B (SEQ ID NO:2) excluding the N-terminal 6 histidines, as shown in FIG. 1C (SEQ ID NO:3), variant TNF-α proteins have at least about 1 residue that differs from the human TNF-α sequence, with at least about 2, 3, 4, 5, 6, 7 or 8 different residues. Preferred variant TNF-α proteins have 3 to 8 different residues.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, “percent (%) nucleic acid sequence identity” with respect to the coding sequence of the polypeptides identified is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

TNF-α proteins may be fused to, for example, other therapeutic proteins or to other proteins such as Fc or serum albumin for therapeutic or pharmacokinetic purposes. In this embodiment, a TNF-α protein implemented in embodiments of the invention is operably linked to a fusion partner. The fusion partner may be any moiety that provides an intended therapeutic or pharmacokinetic effect. Examples of fusion partners include but are not limited to Human Serum Albumin, a therapeutic agent, a cytotoxic or cytotoxic molecule, radionucleotide, and an Fc, etc. As used herein, an Fc fusion is synonymous with the terms “immunoadhesin”, “Ig fusion”, “Ig chimera”, and “receptor globulin” as used in the prior art (Chamow et al., 1996, Trends Biotechnol 14:52-60; Ashkenazi et al., 1997, Curr Opin Immunol 9:195-200, both incorporated by reference). An Fc fusion combines the Fc region of an immunoglobulin with the target-binding region of a TNF-α protein, for example. See for example U.S. Pat. Nos. 5,766,883 and 5,876,969, both of which are hereby incorporated by reference.

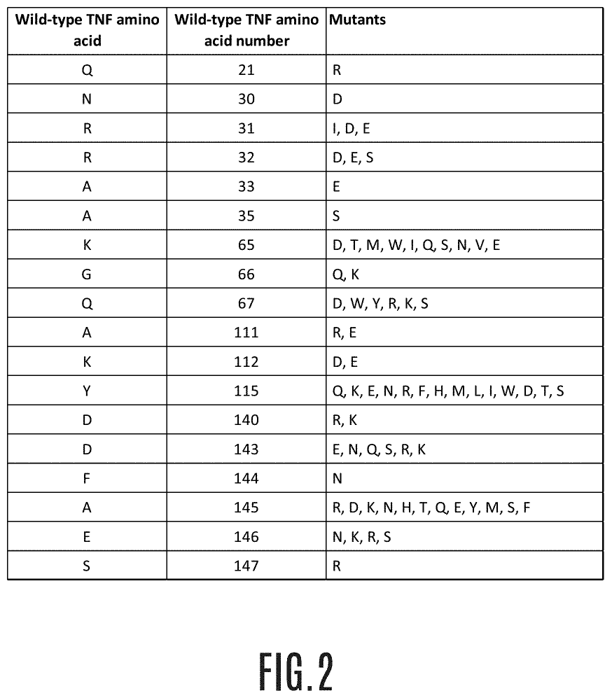

In a preferred embodiment, the variant TNF-α proteins comprise variant residues selected from the following positions 21, 23, 30, 31, 32, 33, 34, 35, 57, 65, 66, 67, 69, 75, 84, 86, 87, 91, 97, 101, 111, 112, 115, 140, 143, 144, 145, 146, and 147. Preferred amino acids for each position, including the human TNF-α residues, are shown in FIG. 2. Thus, for example, at position 143, preferred amino acids are Glu, Asn, Gln, Ser, Arg, and Lys; etc. Preferred changes include: V1M, Q21C, Q21 R, E23C, R31C, N34E, V91E, Q21R, N30D, R31C, R31I, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, I97R, I97T, C101A, A111R, A111E, K112D, K112E, Y115D, Y115E, Y115F, Y115H, Y115I, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R. These may be done either individually or in combination, with any combination being possible. However, as outlined herein, preferred embodiments utilize at least 1 to 8, and preferably more, positions in each variant TNF-α protein.

In an additional aspect, the invention provides TNF-α variants selected from the group consisting of XENP268 XENP344, XENP345, XENP346, XENP550, XENP551, XENP557, XENP1593, XENP1594, and XENP1595 as outlined in Example 3 OF U.S. Pat. No. 7,662,367, which is incorporated herein by reference.

In an additional aspect, the invention makes use of methods of forming a TNF-α heterotrimer in vivo in a mammal comprising administering to the mammal a variant TNF-α molecule as compared to the corresponding wild-type mammalian TNF-α, wherein said TNF-α variant is substantially free of agonistic activity.

In an additional aspect, the invention makes use of methods of screening for selective inhibitors comprising contacting a candidate agent with a soluble TNF-α protein and assaying for TNF-α biological activity; contacting a candidate agent with a transmembrane TNF-α protein and assaying for TNF-α biological activity, and determining whether the agent is a selective inhibitor. The agent may be a protein (including peptides and antibodies, as described herein) or small molecules.

In a further aspect, the invention makes use of variant TNF-α proteins that interact with the wild type TNF-α to form mixed trimers incapable of activating receptor signaling. Preferably, variant TNF-α proteins with 1, 2, 3, 4, 5, 6 and 7 amino acid changes are used as compared to wild type TNF-α protein. In a preferred embodiment, these changes are selected from positions 1, 21, 23, 30, 31, 32, 33, 34, 35, 57, 65, 66, 67, 69, 75, 84, 86, 87, 91, 97, 101, 111, 112, 115, 140, 143, 144, 145, 146 and 147. In an additional aspect, the non-naturally occurring variant TNF-α proteins have substitutions selected from the group of substitutions consisting of: V1M, Q21C, Q21R, E23C, N34E, V91E, Q21R, N30D, R31C, R311, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, I97R, I97T, C101A, A111R, A111E, K112D, K112E, Y115D, Y115E, Y115F, Y115H, Y115I, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R.

In another preferred embodiment, substitutions may be made either individually or in combination, with any combination being possible. Preferred embodiments utilize at least one, and preferably more, positions in each variant TNF-α protein. For example, substitutions at positions 31, 57, 69, 75, 86, 87, 97, 101, 115, 143, 145, and 146 may be combined to form double variants. In addition, triple, quadruple, quintuple and the like, point variants may be generated.

In one aspect the invention makes use of TNF-α variants comprising the amino acid substitutions A145R/I97T. In one aspect, the invention provides TNF-α variants comprising the amino acid substitutions V1M, R31C, C69V, Y87H, C101A, and A145R. In a preferred embodiment, this variant is PEGylated.

In a preferred embodiment the variant is XPRO1595, a PEGylated protein comprising V1M, R31C, C69V, Y87H, C101A, and A145R mutations relative to the wild type human sequence, also referred to herein as “XPro”.

For purposes of the present invention, the areas of the wild type or naturally occurring TNF-α molecule to be modified are selected from the group consisting of the Large Domain (also known as II), Small Domain (also known as I), the DE loop, and the trimer interface. The Large Domain, the Small Domain and the DE loop are the receptor interaction domains. The modifications may be made solely in one of these areas or in any combination of these areas. The Large Domain preferred positions to be varied include: 21, 30, 31, 32, 33, 35, 65, 66, 67, 111, 112, 115, 140, 143, 144, 145, 146 and/or 147. For the Small Domain, the preferred positions to be modified are 75 and/or 97. For the DE Loop, the preferred position modifications are 84, 86, 87 and/or 91. The Trimer Interface has preferred double variants including positions 34 and 91 as well as at position 57. In a preferred embodiment, substitutions at multiple receptor interaction and/or trimerization domains may be combined. Examples include, but are not limited to, simultaneous substitution of amino acids at the large and small domains (e.g. A145R and I97T), large domain and DE loop (A145R and Y87H), and large domain and trimerization domain (A145R and L57F). Additional examples include any and all combinations, e.g., I97T and Y87H (small domain and DE loop). More specifically, theses variants may be in the form of single point variants, for example K112D, Y115K, Y115I, Y115T, A145E or A145R. These single point variants may be combined, for example, Y115I and A145E, or Y1151 and A145R, or Y115T and A145R or Y115I and A145E; or any other combination.

Preferred double point variant positions include 57, 75, 86, 87, 97, 115, 143, 145, and 146; in any combination. In addition, double point variants may be generated including L57F and one of Y115I, Y115Q, Y115T, D143K, D143R, D143E, A145E, A145R, E146K or E146R. Other preferred double variants are Y115Q and at least one of D143N, D143Q, A145K, A145R, or E146K; Y115M and at least one of D143N, D143Q, A145K, A145R or E146K; and L57F and at least one of A145E or 146R; K65D and either D143K or D143R, K65E and either D143K or D143R, Y115Q and any of L75Q, L57W, L57Y, L57F, I97R, I97T, S86Q, D143N, E146K, A145R and I97T, A145R and either Y87R or Y87H; N34E and V91E; L75E and Y115Q; L75Q and Y115Q; L75E and A145R; and L75Q and A145R.

Further, triple point variants may be generated. Preferred positions include 34, 75, 87, 91, 115, 143, 145 and 146. Examples of triple point variants include V91 E, N34E and one of Y115I, Y115T, D143K, D143R, A145R, A145E E146K, and E146R. Other triple point variants include L75E and Y87H and at least one of Y115Q, A145R, Also, L75K, Y87H and Y115Q. More preferred are the triple point variants V91E, N34E and either A145R or A145E.

Variant TNF-α proteins may also be identified as being encoded by variant TNF-α nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred. In a preferred embodiment, a variant TNF-α nucleic acid encodes a variant TNF-α protein. As will be appreciated by those in the art, due to the degeneracy of the genetic code, an extremely large number of nucleic acids may be made, all of which encode the variant TNF-α proteins of the present invention. Thus, having identified a particular amino acid sequence, those skilled in the art could make any number of different nucleic acids, by simply modifying the sequence of one or more codons in a way which does not change the amino acid sequence of the variant TNF-α.

In one embodiment, the nucleic acid homology is determined through hybridization studies. Thus, for example, nucleic acids which hybridize under high stringency to the nucleic acid sequence shown in FIG. 1A (SEQ ID NO:1) or its complement and encode a variant TNF-α protein is considered a variant TNF-α gene. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), incorporated by reference. Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. In another embodiment, less stringent hybridization conditions are used; for example, moderate or low stringency conditions may be used, as are known in the art; see Maniatis and Ausubel, supra, and Tijssen, supra. In addition, nucleic acid variants encode TNF-α protein variants comprising the amino acid substitutions described herein. In one embodiment, the TNF-α variant encodes a polypeptide variant comprising the amino acid substitutions A145R/197T. In one aspect, the nucleic acid variant encodes a polypeptide comprising the amino acid substitutions V1M, R31C, C69V, Y87H, C101A, and A145R, or any 1, 2, 3, 4 or 5 of these variant amino acids.

The variant TNF-α proteins and nucleic acids of the present invention are recombinant. As used herein, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments. The nucleic acid may be double stranded, single stranded, or contain portions of both double stranded or single stranded sequence. As will be appreciated by those in the art, the depiction of a single strand (“Watson”) also defines the sequence of the other strand (“Crick”); thus, the sequence depicted in FIG. 1A (SEQ ID NO:1) also includes the complement of the sequence. By the term “recombinant nucleic acid” is meant nucleic acid, originally formed in vitro, in general, by the manipulation of nucleic acid by endonucleases, in a form not normally found in nature. Thus, an isolated variant TNF-α nucleic acid, in a linear form, or an expression vector formed in vitro by ligating DNA molecules that are not normally joined, are both considered recombinant for the purposes of this invention.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a variant TNF-α protein from one organism in a different organism or host cell. Alternatively, the protein may be made at a significantly higher concentration than is normally seen, through the use of an inducible promoter or high expression promoter, such that the protein is made at increased concentration levels. Furthermore, all of the variant TNF-α proteins outlined herein are in a form not normally found in nature, as they contain amino acid substitutions, insertions and deletions, with substitutions being preferred, as discussed below.

Also included within the definition of variant TNF-α proteins of the present invention are amino acid sequence variants of the variant TNF-α sequences outlined herein and shown in the Figures. That is, the variant TNF-α proteins may contain additional variable positions as compared to human TNF-α. These variants fall into one or more of three classes: substitutional, insertional or deletional variants.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Using the nucleic acids disclosed herein, which encode a variant TNF-α protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the variant TNF-α protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

In a preferred embodiment, when the endogenous secretory sequence leads to a low level of secretion of the naturally occurring protein or of the variant TNF-α protein, a replacement of the naturally occurring secretory leader sequence is desired. In this embodiment, an unrelated secretory leader sequence is operably linked to a variant TNF-α encoding nucleic acid leading to increased protein secretion. Thus, any secretory leader sequence resulting in enhanced secretion of the variant TNF-α protein, when compared to the secretion of TNF-α and its secretory sequence, is desired. Suitable secretory leader sequences that lead to the secretion of a protein are known in the art. In another preferred embodiment, a secretory leader sequence of a naturally occurring protein or a protein is removed by techniques known in the art and subsequent expression results in intracellular accumulation of the recombinant protein.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used. A preferred expression vector system is a retroviral vector system such as is generally described in PCT/US97/01019 and PCT/US97/01048, both of which are hereby incorporated by reference. In a preferred embodiment, the expression vector comprises the components described above and a gene encoding a variant TNF-α protein. As will be appreciated by those in the art, all combinations are possible and accordingly, as used herein, the combination of components, comprised by one or more vectors, which may be retroviral or not, is referred to herein as a “vector composition”.

A number of viral based vectors have been used for gene delivery. See for example U.S. Pat. No. 5,576,201, which is expressly incorporated herein by reference. For example, retroviral systems are known and generally employ packaging lines which have an integrated defective provirus (the “helper”) that expresses all of the genes of the virus but cannot package its own genome due to a deletion of the packaging signal, known as the psi sequence. Thus, the cell line produces empty viral shells. Producer lines can be derived from the packaging lines which, in addition to the helper, contain a viral vector, which includes sequences required in cis for replication and packaging of the virus, known as the long terminal repeats (LTRs). The gene of interest can be inserted in the vector and packaged in the viral shells synthesized by the retroviral helper. The recombinant virus can then be isolated and delivered to a subject. (See, e.g., U.S. Pat. No. 5,219,740.) Representative retroviral vectors include but are not limited to vectors such as the LHL, N2, LNSAL, LSHL and LHL2 vectors described in e.g., U.S. Pat. No. 5,219,740, incorporated herein by reference in its entirety, as well as derivatives of these vectors. Retroviral vectors can be constructed using techniques well known in the art. See, e.g., U.S. Pat. No. 5,219,740; Mann et al. (1983) Cell 33:153-159.

Adenovirus based systems have been developed for gene delivery and are suitable for delivery according to the methods described herein. Human adenoviruses are double-stranded DNA viruses that enter cells by receptor-mediated endocytosis. These viruses are particularly well suited for gene transfer because they are easy to grow and manipulate and they exhibit a broad host range in vivo and in vitro.

Adenoviruses infect quiescent as well as replicating target cells. Unlike retroviruses which integrate into the host genome, adenoviruses persist extrachromosomally thus minimizing the risks associated with insertional mutagenesis. The virus is easily produced at high titers and is stable so that it can be purified and stored. Even in the replication-competent form, adenoviruses cause only low-level morbidity and are not associated with human malignancies. Accordingly, adenovirus vectors have been developed which make use of these advantages. For a description of adenovirus vectors and their uses see, e.g., Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; Rich et al. (1993) Human Gene Therapy 4:461-476.

In a preferred embodiment, the viral vectors used in the subject methods are AAV vectors. By an “AAV vector” is meant a vector derived from an adeno-associated virus serotype, including without limitation, AAV-1, AAV-2, AAV-3, AAV-4, AAV-5, AAVX7, etc. Typical AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. An AAV vector includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. For more on various AAV serotypes, see for example Cearley et al., Molecular Therapy, 16:1710-1718, 2008, which is expressly incorporated herein in its entirety by reference.

AAV expression vectors may be constructed using known techniques to provide as operatively linked components in the direction of transcription, control elements including a transcriptional initiation region, the DNA of interest and a transcriptional termination region. The control elements are selected to be functional in a thalamic and/or cortical neuron. Additional control elements may be included. The resulting construct, which contains the operatively linked components is bounded (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAVX7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

Suitable DNA molecules for use in AAV vectors will include, for example, a gene that encodes a protein that is defective or missing from a recipient subject or a gene that encodes a protein having a desired biological or therapeutic effect (e.g., an enzyme, or a neurotrophic factor). The artisan of reasonable skill will be able to determine which factor is appropriate based on the neurological disorder being treated.

The selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available.

Once made, the TNF-α protein may be covalently modified. For instance, a preferred type of covalent modification of variant TNF-α comprises linking the variant TNF-α polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337, incorporated by reference. These nonproteinaceous polymers may also be used to enhance the variant TNF-α's ability to disrupt receptor binding, and/or in vivo stability. In another preferred embodiment, cysteines are designed into variant or wild type TNF-α in order to incorporate (a) labeling sites for characterization and (b) incorporate PEGylation sites. For example, labels that may be used are well known in the art and include but are not limited to biotin, tag and fluorescent labels (e.g. fluorescein). These labels may be used in various assays as are also well known in the art to achieve characterization. A variety of coupling chemistries may be used to achieve PEGylation, as is well known in the art. Examples include but are not limited to, the technologies of Shearwater and Enzon, which allow modification at primary amines, including but not limited to, lysine groups and the N-terminus See, Kinstler et al, Advanced Drug Deliveries Reviews, 54, 477-485 (2002) and M J Roberts et al, Advanced Drug Delivery Reviews, 54, 459-476 (2002), both hereby incorporated by reference.

In one preferred embodiment, the optimal chemical modification sites are 21, 23, 31 and 45, taken alone or in any combination. In an even more preferred embodiment, a TNF-α variant of the present invention includes the R31C mutation.

In a preferred embodiment, the variant TNF-α protein is purified or isolated after expression. Variant TNF-α proteins may be isolated or purified in a variety of ways known to those skilled in the art depending on what other components are present in the sample.

In another preferred embodiment, the TNF-α protein is administered via gene modified autologous or allogeneic cellular therapy, wherein the gene therapy comprises mesenchymal stem cells expressing a construct of the TNF-α protein, preferably a DN-TNF-α protein, more preferably XPRO1595.

### Treatment Methods

The terms “treatment”, “treating”, and the like, as used herein include amelioration or elimination of a disease or condition once it has been established or alleviation of the characteristic symptoms of such disease or condition. A method as disclosed herein may also be used to, depending on the condition of the patient, prevent the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition as described herein to a patient that is not at the time of administration afflicted with the disease or condition. “Preventing” also encompasses preventing the recurrence or relapse-prevention of a disease or condition or of symptoms associated therewith, for instance after a period of improvement.

In one embodiment, a selective inhibitor of solTNF-α as described herein is administered peripherally to a patient in need thereof to reduce inflammation and/or reduce NAS and/or reduce fibrosis.

In one embodiment, the treatment method includes administering a selective inhibitor of solTNF-α as described herein to a patient diagnosed with NASH. Prior to or subsequent to treatment, the patient may be initially selected, or post-treatment monitored for improvements, by measuring a number of biomarkers, including levels of: adiponectin (ADP), tumor necrosis factor alpha (TNF-α), leptin, c-reactive Protein (CRP), interleukin-6 (IL-6), oxidized low-density lipoprotein OxLDL), lipoprotein receptor-1 (LOX-1), interleukin-17 (IL-17), cytokeratin 18 (CK18) whole protein, cytokeratin 18 (CK18) caspase-cleaved fragments, soluble Fas (sFas), soluble Fas ligand (sFasL), ferritin, and/or blood neutrophil to lymphocyte (N/L) ratio in accordance with techniques known to one having skill in the art. Additionally, or alternatively, the patient may be monitored for improvements by measuring a degree of fibrosis. While liver biopsy remains the gold standard for NASH diagnosis as of the date of this disclosure, other non-invasive diagnostics are in development. Examples of methods for non-invasive diagnosis of NASH will be described in further detail below.

In one embodiment, the method may comprise subcutaneous injection of the selective inhibitor of solTNF-α for treatment of NASH, hepatic steatosis; non-alcoholic hepatic steatosis; fibrotic liver disease, including cirrhosis secondary to chronic inflammatory disease; and intestinal inflammation.

In an alternative embodiment the method may comprise topical administration of a selective inhibitor of solTNF-α as described herein. In this embodiment the DN-TNF-α may be formulated as a lotion or cream.

Other methods of administration are further described herein.

### Formulations

Depending upon the manner of introduction, the pharmaceutical composition may be formulated in a variety of ways. The concentration of the therapeutically active variant TNF-α protein in the formulation may vary from about 0.1 to 100 weight %. In another preferred embodiment, the concentration of the variant TNF-α protein is in the range of 0.003 to 1.0 molar, with dosages from 0.03, 0.05, 0.1, 0.2, and 0.3 millimoles per kilogram of body weight being preferred.

The pharmaceutical compositions for use in embodiments of the present invention comprise a variant TNF-α protein in a form suitable for administration to a patient. In the preferred embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, hereby incorporated by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be.

In one embodiment provided herein, antibodies, including but not limited to monoclonal and polyclonal antibodies, are raised against variant TNF-α proteins using methods known in the art. In a preferred embodiment, these anti-variant TNF-α antibodies are used for immunotherapy. Thus, methods of immunotherapy are provided. By “immunotherapy” is meant treatment of TNF-α related disorders with an antibody raised against a variant TNF-α protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant TNF-α protein antigen to which antibodies are raised. As appreciated by one of ordinary skill in the art, the variant TNF-α protein antigen may be provided by injecting a variant TNF-α polypeptide against which antibodies are desired to be raised into a recipient, or contacting the recipient with a variant TNF-α protein encoding nucleic acid, capable of expressing the variant TNF-α protein antigen, under conditions for expression of the variant TNF-α protein antigen.

In another preferred embodiment, a therapeutic compound is conjugated to an antibody, preferably an anti-variant TNF-α protein antibody. The therapeutic compound may be a cytotoxic agent. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against cell cycle proteins, or binding of a radionuclide to a chelating agent that has been covalently attached to the antibody.

In a preferred embodiment, variant TNF-α proteins are administered as therapeutic agents, and can be formulated as outlined above. Similarly, variant TNF-α genes (including both the full-length sequence, partial sequences, or regulatory sequences of the variant TNF-α coding regions) may be administered in gene therapy applications, as is known in the art. These variant TNF-α genes can include antisense applications, either as gene therapy (i.e. for incorporation into the genome) or as antisense compositions, as will be appreciated by those in the art.

In a preferred embodiment, the nucleic acid encoding the variant TNF-α proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. U.S.A. 83:4143-4146 (1986), incorporated by reference). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993), incorporated by reference). In some situations, it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), both incorporated by reference. For review of gene marking and gene therapy protocols see Anderson et al., Science 256:808-813 (1992), incorporated by reference.

In a preferred embodiment, variant TNF-α genes are administered as DNA vaccines, either single genes or combinations of variant TNF-α genes. Naked DNA vaccines are generally known in the art. Brower, Nature Biotechnology, 16:1304-1305 (1998). Methods for the use of genes as DNA vaccines are well known to one of ordinary skill in the art, and include placing a variant TNF-α gene or portion of a variant TNF-α gene under the control of a promoter for expression in a patient in need of treatment. The variant TNF-α gene used for DNA vaccines can encode full-length variant TNF-α proteins, but more preferably encodes portions of the variant TNF-α proteins including peptides derived from the variant TNF-α protein. In a preferred embodiment, a patient is immunized with a DNA vaccine comprising a plurality of nucleotide sequences derived from a variant TNF-α gene. Similarly, it is possible to immunize a patient with a plurality of variant TNF-α genes or portions thereof as defined herein. Without being bound by theory, expression of the polypeptide encoded by the DNA vaccine, cytotoxic T-cells, helper T-cells and antibodies are induced, which recognize and destroy or eliminate cells expressing TNF-α proteins.

In a preferred embodiment, the DNA vaccines include a gene encoding an adjuvant molecule with the DNA vaccine. Such adjuvant molecules include cytokines that increase the immunogenic response to the variant TNF-α polypeptide encoded by the DNA vaccine. Additional or alternative adjuvants are known to those of ordinary skill in the art and find use in the invention.

Pharmaceutical compositions are contemplated wherein a TNF-α variant of the present invention and one or more therapeutically active agents are formulated. Formulations of the present invention are prepared for storage by mixing TNF-α variant having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions. Lyophilization is well known in the art, see, e.g., U.S. Pat. No. 5,215,743, incorporated entirely by reference. Acceptable carriers, excipients, or stabilizers are nontoxic to recipients at the dosages and concentrations employed, and include buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN®, PLURONICS® or polyethylene glycol (PEG). In a preferred embodiment, the pharmaceutical composition that comprises the TNF-α variant of the present invention may be in a water-soluble form. The TNF-α variant may be present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

### Controlled Release

In addition, any of a number of delivery systems are known in the art and may be used to administer TNF-α variants in accordance with embodiments of the present invention. Examples include, but are not limited to, encapsulation in liposomes, microparticles, microspheres (e.g. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT®, and poly-D-(-)-3-hydroxyburyric acid. It is also possible to administer a nucleic acid encoding the TNF-α of the current invention, for example by retroviral infection, direct injection, or coating with lipids, cell surface receptors, or other transfection agents. In all cases, controlled release systems may be used to release the TNF-α at or close to the desired location of action.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, incorporated entirely by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be. The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, incorporated entirely by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be.

Dosage forms for the topical or transdermal administration of a DN-TNF-protein disclosed herein include powders, sprays, ointments, pastes, creams, lotions, gels, solutions, patches and inhalants. The DN-TNF-protein may be mixed under sterile conditions with a pharmaceutically-acceptable carrier, and with any preservatives, buffers, or propellants which may be required. Powders and sprays can contain, in addition to the DN-TNF-protein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

### Methods of Administration

The administration of the selective inhibitor of solTNF in accordance with embodiments of the present invention, preferably in the form of a sterile aqueous solution, is done peripherally, in a variety of ways, including, but not limited to, orally, subcutaneously, intravenously, intranasally, transdermally, intraperitoneally, intramuscularly, intrapulmonary, vaginally, rectally, or intraocularly. In some instances, the selective inhibitor of solTNF may be directly applied as a solution, salve, cream or spray. The selective inhibitor of solTNF may also be delivered by bacterial or fungal expression into the human system (e.g., WO 04046346 A2, hereby incorporated by reference).

### Subcutaneous

Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate. A selective inhibitor of solTNF may be more amenable to subcutaneous administration due to, for example, increased potency, improved serum half-life, or enhanced solubility.

### Intravenous

As is known in the art, protein therapeutics are often delivered by IV infusion or bolus. The selective inhibitor of solTNF may also be delivered using such methods. For example, administration may be by intravenous infusion with 0.9% sodium chloride as an infusion vehicle.

### Inhaled

Pulmonary delivery may be accomplished using an inhaler or nebulizer and a formulation comprising an aerosolizing agent. For example, inhalable technology, or a pulmonary delivery system may be used. The selective inhibitor of solTNF may be more amenable to intrapulmonary delivery. The selective inhibitor of solTNF may also be more amenable to intrapulmonary administration due to, for example, improved solubility or altered isoelectric point.

### Oral Delivery

Furthermore, the selective inhibitor of solTNF may be more amenable to oral delivery due to, for example, improved stability at gastric pH and increased resistance to proteolysis.

### Transdermal

Transdermal patches may have the added advantage of providing controlled delivery of the selective inhibitor of solTNF to the body. Dissolving or dispersing DN-TNF-protein in the proper medium can make such dosage forms. Absorption enhancers can also be used to increase the flux of DN-TNF-protein across the skin. Either providing a rate controlling membrane or dispersing DN-TNF-protein in a polymer matrix or gel can control the rate of such flux.

### Intraocular

Ophthalmic formulations, eye ointments, powders, solutions and the like, are also contemplated as being suitable for use in embodiments of this invention.

In a preferred embodiment, the selective inhibitor of solTNF is administered as a therapeutic agent, and can be formulated as outlined above. Similarly, variant TNF-α genes (including both the full-length sequence, partial sequences, or regulatory sequences of the variant TNF-α coding regions) may be administered in gene therapy applications, as is known in the art. These variant TNF-α genes can include antisense applications, either as gene therapy (i.e. for incorporation into the genome) or as antisense compositions, as will be appreciated by those in the art.

In a preferred embodiment, the nucleic acid encoding the variant TNF-α proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. U.S.A. 83:4143-4146 (1986), incorporated entirely by reference). The oligonucleotides can be modified to enhance their uptake, e.g. by substituting their negatively charged phosphodiester groups by uncharged groups.

### Dosage

Dosage may be determined depending on the complication being treated and mechanism of delivery. Typically, an effective amount of the selective inhibitor of solTNF, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 2000 mg per kilogram body weight per day. An exemplary treatment regime entails administration once every day or once a week or once a month. A DN-TNF protein may be administered on multiple occasions. Intervals between single dosages can be daily, weekly, monthly or yearly. Alternatively, A DN-TNF protein may be administered as a sustained release formulation, in which case less frequent administration is required. Dosage and frequency vary depending on the half-life of the agent in the subject. The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the patent can be administered a prophylactic regime.

### Toxicity

Suitably, an effective amount (e.g., dose) of a DN-TNF protein described herein will provide therapeutic benefit without causing substantial toxicity to the subject. Toxicity of the agent described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the agent described herein lies suitably within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

### Non-Invasive Diagnosis of NASH

### Biomarkers of NASH

### Markers of Apoptosis

Increased cell death in the liver has emerged as an important mechanism that contributes to disease progression to NASH. Programmed cell death, or Apoptosis, is a highly organized process that can occur via two fundamental pathways: extrinsic mediation by death receptors (such as Fas) or intrinsic mediation by organelles (such as mitochondria). Both pathways can lead to the activation of effector caspases (mainly caspase 3), which cleave different intracellular substrates, including cytokeratin 18 (CK18), which is the major intermediate filament protein in hepatocytes. Caspase-generated CK18 fragment levels can be measured in plasma, for example using the M30 monoclonal antibody enzyme-linked immunosorbent assay (ELISA), and also in serum. These levels have been found to be significant in NASH patients.

In contrast to the M30 ELISA, which only detects caspase-cleaved CK18 (CK18 fragments), the M65 ELISA can detect both caspase-cleaved and uncleaved CK18 (total CK18), and this assay is used as a marker of overall hepatocyte death, including both apoptosis and necrosis. In addition, this assay can be used to detect steatosis, steatohepatitis, and liver fibrosis in a cohort of patients with chronic liver disease.

### Markers of Oxidative Stress

Oxidative stress plays a central role in hepatocyte injury and disease progression from Simple Steatosis (SS) to NASH, but precise molecular species have not yet been identified. Several oxidation pathways may play a role in the overproduction of lipid peroxidation products in NASH patients, including enzymatic and nonenzymatic free radical-mediated processes. Each of these pathways may generate different oxidation products that could potentially be quantified. Systemic lipid peroxidation has been measured in patients with biopsy-confirmed NASH and control patients matched by age, gender, and body mass index (BMI); it was previously found that levels of both oxidized low-density lipoprotein (oxLDL) and thiobarbituric acid-reacting substances were significantly higher in NASH patients.

### Markers of Inflammation

The inflammatory state that exists in obesity and NAFLD may contribute to disease progression to NASH. Levels of proinflammatory cytokines, such as tumor necrosis factor-α (TNF-α) and interleukin (IL)-6, have previously been shown to be higher in NASH patients than SS patients. The blood neutrophil to lymphocyte (N/L) ratio is a simple indicator of the overall inflammatory status of the body that has previously been used to predict outcomes in patients with cancer and coronary artery disease. The N/L ratio has been identified as a noninvasive marker of NAFLD severity, and it has been previously demonstrated that the N/L ratio was higher in patients with NASH than those with SS.

Furthermore, the N/L ratio has been shown to correlate with the main histologic features of NAFLD, including inflammation and fibrosis.

Serum ferritin is an acute-phase reactant that can be induced in the setting of chronic systemic inflammation, and it has been observed to be elevated in patients with obesity-related complications such as diabetes and metabolic syndrome (MetS). It has been demonstrated that ferritin levels more than 1.5 times the upper limit of normal were associated with the diagnosis of NASH and advanced fibrosis in a large cohort of biopsy-confirmed NAFLD patients who were enrolled in the NASH Clinical Research Network (CRN).

### Predictive Models

Predictive models combine routinely assessed clinical variables with laboratory tests and biomarkers (such as hepatocyte apoptosis markers, oxidative stress markers, and inflammatory cytokines) to accurately predict the presence of NASH on liver biopsy. Examples of predictive models that use a combination of clinical and laboratory data are the HAIR score (which is based on hypertension, alanine aminotransferase (ALT) level, and insulin resistance) and the NASH predictive index (which is based on age, gender, BMI, homeostatic model assessment of insulin resistance, and log [aspartate aminotransferase {AST}×ALT]). The NASHTest (BioPredictive) was developed in a set of 160 patients by combining 13 clinical and biochemical variables: age; gender; weight; height; and serum levels of cholesterol, triglycerides, α2 macroglobulin, apolipoprotein A1, haptoglobin, gamma glutamyltransferase (GGT), ALT, AST, and bilirubin. The NASHTest has been validated in a cohort of 97 patients from different centers.

The NASH CRN recently developed progressive models based on readily available clinical and laboratory variables for predicting histologic diagnoses on liver biopsy (including the presence of NASH). A model based on AST level, ALT level, AST/ALT ratio, demographics (age, race, gender, and ethnicity), comorbidities (hypertension, type 2 diabetes, BMI, waist circumference, waist/hip ratio, and Acanthosis nigricans), and other laboratory tests was validated for predicting NASH on liver biopsy; in addition, this model was validated for predicting the presence of ballooning degeneration, which is a main histologic feature of NASH.

Several predictive models include NASH biomarkers in addition to clinical variables. For example, CK18 has been combined with ALT levels and the presence of MetS in a new composite scoring system (known as the Nice model) designed to diagnose NASH in morbidly obese patients; this system has yielded promising results. Others have developed a NAFLD diagnostic panel (with a NASH prediction model) based on diabetes, gender, BMI, triglycerides, M30 (CK18 fragments as a marker of apoptosis), and M65 plus M30 (total CK18 and CK18 fragments as a marker of necrosis).

The risk score oxNASH is based on multivariable modeling and the finding that products of free radical-mediated oxidation of linoleic acid were significantly higher in patients with NASH. This score was calculated from age, BMI, AST level, and the ratio of 13-hydroxy octadecadienoic acid to linoleic acid. Patients with oxNASH scores over 72 were 10 times more likely to have NASH than patients with oxNASH scores less than 47. In a sample of 122 patients with biopsy-confirmed NAFLD, it was previously demonstrated that oxNASH scores correlated with histologic features that define NASH, including steatosis, ballooning, and inflammation.

### Non-Invasive Diagnosis of Liver Fibrosis

The presence and extent of fibrosis is an important factor in the prognosis of NAFLD and in the prediction of the risk of progression to cirrhosis and its complications. Factors that predict the development of progressive fibrosis and cirrhosis include obesity, type 2 diabetes, age older than 45 years, an elevated AST/ALT ratio, hypertension, and hyperlipidemia. Over the past decade, many noninvasive strategies have been developed to predict the stage of liver fibrosis in this patient population. Non-radiologic tests can be grouped into simple bedside models (which use a combination of age, BMI, AST/ALT ratio, and other clinical variables) or more complex models such as the Enhanced Liver Fibrosis (ELF) panel (which use serum markers of fibrosis). The stages of NASH-associated fibrosis range from absent (stage F0) to cirrhosis (stage 4), with stages F2-F4 considered to be clinically significant and stages F3-F4 considered to be advanced fibrosis.

### Simple Predictive Models for Advanced Fibrosis

### AST/ALT Ratio

ALT levels are usually higher than AST levels in NAFLD patients; however, an AST/ALT ratio greater than 1 is suggestive of an advanced fibrotic form of the disease. This ratio is the simplest predictive model for advanced fibrosis, and it can be calculated using two readily available liver function tests. Despite its simplicity, this ratio has a good negative predictive value and can be used to rule out the presence of advanced fibrosis. The AST/ALT ratio has also been incorporated into other models, including the BMI, AST/ALT ratio, and diabetes (BARD) score and the NAFLD fibrosis score (NFS).

### BMI, AST/ALT Ratio, and Diabetes (BARD) Score

This score combines three variables in a weighted sum in order to generate an easily calculated composite score for predicting advanced fibrosis (BMI≥28=1 point; AST/ALT ratio≥0.8=2 points; and the presence of diabetes=1 point). A BARD score of at least 2 was associated with stages 3-4 fibrosis.

### Nonalcoholic Fatty Liver Disease Fibrosis Score

The NFS is based on age, hyperglycemia, BMI, platelet count, albumin level, and AST/ALT ratio. The score has two cutoff values: a score of less than −1.455 predicts the absence of advanced fibrosis, whereas a score greater than 0.675 predicts the presence of advanced fibrosis. The NFS has been validated in multiple studies. The recent NAFLD guidelines acknowledged that the NFS is a clinically useful tool for identifying advanced fibrosis in patients with NAFLD. This score is available online at http://nafldscore.com/, and it can be easily calculated during patient visits.

### FIB4 Index

The FIB4 index was originally developed to stage liver fibrosis in patients with hepatitis C virus infection; this index is based on age, platelet count, ALT level, and AST level. The FIB4 index has been used in NAFLD patients. Using a cutoff value less than 1.3, the FIB4 index has a negative predictive value of 90-95% for ruling out advanced fibrosis.

### Nonalcoholic Steatohepatitis Clinical Research Network Model

The NASH CRN model is based on AST level, ALT level, AST/ALT ratio, demographic factors, comorbidities, and other laboratory test results. This model has been validated for predicting advanced fibrosis (stages F3-F4) and for predicting cirrhosis (stage F4) on liver biopsy.

### Complex Predictive Models Using Biomarkers of Fibrosis Enhanced Liver Fibrosis Panel

This panel is based on the idea that liver fibrosis is a dynamic process that results in increased plasma levels of markers of extracellular matrix turnover. The panel includes three biomarkers of fibrosis (hyaluronic acid, tissue inhibitor of metalloproteinase 1, and amino-terminal peptide of procollagen III), and the panel is excellent at detecting advanced fibrosis. Combining the ELF panel with the NFS increased diagnostic accuracy for fibrosis stages F3-F4. The ELF panel was previously shown to be a good predictor of clinical outcomes (liver-related morbidity and mortality) in a group of patients with chronic liver disease, including forty-four patients with NAFLD, making the panel a promising prognostic tool.

### FibroTest

FibroTest (BioPredictive) is a panel that uses five biomarkers—haptoglobin, alpha-2-macroglobulin, apo lipoprotein A1, total bilirubin, and GGT—to predict the presence of fibrosis.

### Exemplary Features and Embodiments of the Invention

Certain preferred embodiments can be summarized as follows:

A method is disclosed for treating a subject diagnosed with NAFLD and/or NASH, the method comprising: administering to the subject a therapeutically effective amount of a selective inhibitor of solTNF-α, whereby the subject is treated; for purposes herein, this shall constitute “the method”.

The selective inhibitor of solTNF-α may comprise a DN-TNF-α protein or a nucleic acid encoding the DN-TNF-α protein.

The DN-TNF-α protein may comprise XPRO1595. Thus, in an embodiment, the method may comprise administering XPRO1595 in a dose between 0.1 mg/kg and 10.0 mg/kg.

The DN-TNF-α protein can be administered: intravenously; subcutaneously; orally; via aerosol; via topical application; or via gene therapy. The gene therapy may comprise mesenchymal stem cells expressing a construct of the DN-TNF-α protein. The DN-TNF-α protein can be administered via gene modified autologous or allogeneic cellular therapy.

The method may further comprise: measuring in the subject at least one biomarker of NASH, wherein said biomarker of NASH is selected from the group consisting of: adiponectin (ADP), tumor necrosis factor alpha (TNF-α), leptin, c-reactive Protein (CRP), interleukin-6 (IL-6), oxidized low-density lipoprotein OxLDL), lipoprotein receptor-1 (LOX-1), interleukin-17 (IL-17), cytokeratin 18 (CK18) whole protein, cytokeratin 18 (CK18) caspase-cleaved fragments, soluble Fas (sFas), soluble Fas ligand (sFasL), ferritin, and blood neutrophil to lymphocyte (N/L) ratio; and if the at least one biomarker measured exceeds (goes beyond; i.e. greater than or less than) normal threshold, then performing the step of administering to the subject a therapeutically effective amount of a selective inhibitor of solTNF-α. The following Table 0.1 illustrates the current accepted normal threshold for each of the NASH biomarkers in the proposed group; however, it should be recognized that the accepted normal threshold can vary as technologies are advanced and knowledge in the art is further developed.

TABLE 0.1
Biomarkers of NASH and corresponding Normal Threshold

| Biomarker of NASH | Normal Threshold | |
|---|---|---|
| adiponectin (ADP) | >8.0 | ng/mL (serum) |
| tumor necrosis factor alpha (TNF-α) | <3.0 | pg/mL (serum) |
| leptin | <20.0 | ng/mL (serum) |
| c-reactive Protein (CRP) | <5.0 | mg/L (serum) |
| interleukin-6 (IL-6) | <5.0 | pg/mL (serum) |
| oxidized low-density lipoprotein (OxLDL) | <90.0 | U/L (serum) |
| lipoprotein receptor-1 (LOX-1) | <5.0 | ng/mL (serum) |
| interleukin-17 (IL-17) | <8.0 | pg/mL (serum) |
| cytokeratin 18 (CK18) whole protein | <300 | U/L (serum) |
| cytokeratin 18 (CK18) caspase-cleaved | <200 | U/L (serum) |
| fragments | | |
| soluble Fas (sFas) | <600 | pg/mL (serum) |
| soluble Fas ligand (sFasL) | <250.0 | pg/mL (serum) |
| ferritin | <25.0 | pmol/L (serum) |

| | |
|---|---|
| blood neutrophil to lymphocyte (N/L) ratio | <2.0 |

The method may further comprise: measuring in the subject a NAFLD Activity Score (NAS); and if the NAS measured is greater than or equal to 5, then performing the step of administering to the subject a therapeutically effective amount of a selective inhibitor of solTNF-α.

The method may further comprise: measuring NASH-associated fibrosis in the subject; and if the degree of NASH-associated fibrosis measured is greater than or equal to F2, then performing the step of administering to the subject a therapeutically effective amount of a selective inhibitor of solTNF-α.

### EXAMPLES

### Example 1: Effects of XPRO1595 in STAM Model of NASH

### Materials and Methods

Compound [XPro1595] was provided by INmune Bio International Limited. To prepare dosing solution, Compound was diluted in Vehicle [normal saline].

NASH was induced in 16 male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Japan) after 4 weeks of age.

Compound was administered subcutaneously in a volume of 5 mL/kg.

Compound was administered at dose of 10 mg/kg twice weekly from 8 to 12 weeks of age.

C57BL/6 mice (14-day-pregnant female) were obtained from Japan SLC, Inc. (Japan). All animals used in the study were housed and cared for in accordance with the Japanese Pharmacological Society Guidelines for Animal Use.

The animals were maintained in a SPF facility under controlled conditions of temperature (23±2° C.), humidity (45±10%), lighting (12-hour artificial light and dark cycles; light from 8:00 to 20:00) and air exchange. A high pressure was maintained in the experimental room to prevent contamination of the facility.

The animals were housed in TPX cages (CLEA Japan) with a maximum of 4 mice per cage. Sterilized Paper-Clean (Japan SLC) was used for bedding and replaced once a week.

Sterilized solid HFD was provided ad libitum, being placed in a metal lid on the top of the cage. Pure water was also provided ad libitum from a water bottle equipped with a rubber stopper and a sipper tube. Water bottles were replaced once a week, cleaned, and sterilized in an autoclave and reused.

Mice were identified by ear punch. Each cage was labeled with a specific identification code.

For plasma biochemistry, non-fasting blood was collected in polypropylene tubes with anticoagulant (Novo-Heparin, Mochida Pharmaceutical Co. Ltd., Japan) and centrifuged at 1,000×g for 15 minutes at 4° C. The supernatant was collected and stored at −80° C. until use. Plasma ALT was measured by FUJI DRI-CHEM 7000 (Fujifilm, Japan).

Liver total lipid-extracts were obtained by Folch's method (Folch J. et al., J. Biol. Chem. 1957;226: 497). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride content was measured by Triglyceride E-test (Wako Pure Chemical Industries, Ltd., Japan).

For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner DE. et al., Hepatology, 2005; 41:1313).

To visualize collagen deposition, Bouin's fixed liver sections were stained using picro-Sirius red solution (Waldeck, Germany).

For quantitative analysis of fibrosis area, bright field images of Sirius red-stained section were captured around the central vein using a digital camera (DFC295; Leica, Germany) at 200-fold magnification, and the positive areas in 5 fields/section were measured using ImageJ software (National Institute of Health, USA).

Total RNA was extracted from liver samples using RNAiso (Takara Bio, Japan) according to the manufacturer's instructions. One μg of RNA was reverse-transcribed using a reaction mixture containing 4.4 mM MgCl2 (F. Hoffmann-La Roche, Switzerland), 40 U RNase inhibitor (Toyobo, Japan), 0.5 mM dNTP (Promega, USA), 6.28 μM random hexamer (Promega), 5× first strand buffer (Promega), 10 mM dithiothreitol (Invitrogen, USA) and 200 U MMLV-RT (Invitrogen) in a final volume of 20 μL. The reaction was carried out for 1 hour at 37° C., followed by 5 minutes at 99° C. Real-time PCR was performed using real-time PCR DICE and TB Green™ Premix Ex Taq™ II (Takara Bio). To calculate the relative mRNA expression level, the expression of each gene (TNF-α, IFN-γ, Collagen Type 1, TGF-β, TIMP-1 and MCP-1) was normalized to that of reference gene 36B4 (gene symbol: Rplp0). Information of PCR-primer sets is described in Table 1.1 and the plate layout is described in Table 1.2.

TABLE 1.1
Information of PCR primers

| Gene | Set ID | Sequence |
|---|---|---|
| 36B4 | MA057856 | forward 5′-TTCCAGGCTTTGGGCATCA-3′ |
| | | reverse 5′-ATGTTCAGCATGTTCAGCAGTGTG-3′ |
| TNF-α | MA117190 | forward 5′-TATGGCCCAGACCCTCACA-3′ |
| | | reverse 5′-GGAGTAGACAAGGTACAACCCATC-3′ |
| IFN-γ | MA025911 | forward 5′-CGGCACAGTCATTGAAAGCCTA-3′ |
| | | reverse 5′-GTTGCTGATGGCCTGATTGTC-3′ |
| Collagen Type 1 | MA075477 | forward 5′-CCAACAAGCATGTCTGGTTAGGAG-3′ |
| | | reverse 5′-GCAATGCTGTTCTTGCAGTGGTA-3′ |
| TGF-β | MA030397 | forward 5′-GTGTGGAGCAACATGTGGAACTCTA-3′ |
| | | reverse 5′-TTGGTTCAGCCACTGCCGTA-3′ |
| TIMP-1 | MA098519 | forward 5′-TGAGCCCTGCTCAGCAAAGA-3′ |
| | | reverse 5′-GAGGACCTGATCCGTCCACAA-3′ |
| MCP-1 | MA066003 | forward 5′-GCATCCACGTGTTGGCTCA-3′ |
| | | reverse 5′-CTCCAGCCTACTCATTGGGATCA-3′ |

36B4: Ribosomal protein, large, P0 (Rplp0)
TNF-α: Tumor necrosis factor (Tnf)
IFN-γ: Interferon gamma (Ifng)
Collagen Type 1: Collagen, type I, alpha 2 (Col1a2)
TGF-β: Transforming growth factor, beta 1 (Tgfb1)
TIMP-1: Tissue inhibitor of metalloproteinase 1 (Timp1)
MCP-1: Chemokine (C-C motif) ligand 2 (Ccl2)

TABLE 1.2
Information of PCR-plate

| | Plate |
|---|---|
| | 1 |
| | Mouse ID |
| | 101-208 |
| TNF-α | Plate 1-2 |
| 36B4 | Plate 1-1 |
| IFN-γ | Plate 1-3 |
| 36B4 | Plate 1-1 |
| Collagen Type 1 | Plate 1-4 |
| 36B4 | Plate 1-1 |
| TGF-β | Plate 1-5 |
| 36B4 | Plate 1-1 |
| TIMP-1 | Plate 1-6 |
| 36B4 | Plate 1-1 |
| MCP-1 | Plate 1-7 |
| 36B4 | Plate 1-1 |

For plasma samples, 100 μL of non-fasting blood was collected in polypropylene tubes with anticoagulant (Novo-Heparin) and centrifuged at 1,000×g for 15 minutes at 4° C. The supernatant was collected and stored at −80° C. for biochemistry.

For serum samples, non-fasting blood was collected in serum separate tubes (AS ONE, Japan) without anticoagulant through direct cardiac puncture and centrifuged at 3,500×g for 5 minutes at 4° C. The supernatant was collected and stored at −80° C. for shipping.

For liver samples, left lateral lobe was collected and cut into six pieces. Two pieces of left lateral lobe were fixed in Bouin's solution and then embedded in paraffin. Paraffin blocks were stored at room temperature for histological analyses. The other two pieces of left lateral lobe were embedded in O.C.T. compound and quick frozen in liquid nitrogen. O.C.T. blocks were stored at −80° C. for shipping. The remaining pieces of left lateral lobe were snap frozen in liquid nitrogen and stored at −80° C. for gene expression assay. Right lobe was snap frozen in liquid nitrogen and stored at −80° C. for liver biochemistry. Left and right medial lobes, and caudate lobe were snap frozen in liquid nitrogen and stored at −80° C. for shipping.

Statistical analyses were performed using Student's t-test on GraphPad Prism 6 (GraphPad Software Inc., USA). P values<0.05 were considered statistically significant. A trend or tendency was assumed when a one-tailed t-test returned P values<0.1. Results were expressed as mean±SD.

### Experimental Design and Treatment

Study Group 1 (Vehicle): Eight NASH mice were subcutaneously administered Vehicle [normal saline] in a volume of 5 mL/kg twice weekly from 8 to 12 weeks of age.

Study Group 2 (Compound): Eight NASH mice were subcutaneously administered Vehicle supplemented with Compound at a dose of 10 mg/kg twice weekly from 8 to 12 weeks of age.

TABLE 1.3
Summary of Treatment Schedule

| | No. | | Test | Dose | Volume | | Sacrifice |
|---|---|---|---|---|---|---|---|
| Group | mice | Mice | substance | (mg/kg) | (mL/kg) | Regimen | (wks) |
| 1 | 8 | STAM | Vehicle | — | 5 | SC, BIW, | 12 |
| | | | | | | 8 wks-12 wks | |
| 2 | 8 | STAM | Compound | 10 | 5 | SC, BIW, | 12 |
| | | | | | | 8 wks-12 wks | |

The viability, clinical signs and behavior were monitored daily. Body weight was measured daily during the treatment period. Mice were observed for significant clinical signs of toxicity, moribundity and mortality approximately 60 minutes after each administration. The animals were sacrificed at 12 weeks of age by exsanguination through direct cardiac puncture under isoflurane anesthesia (Pfizer Inc.).

### Results

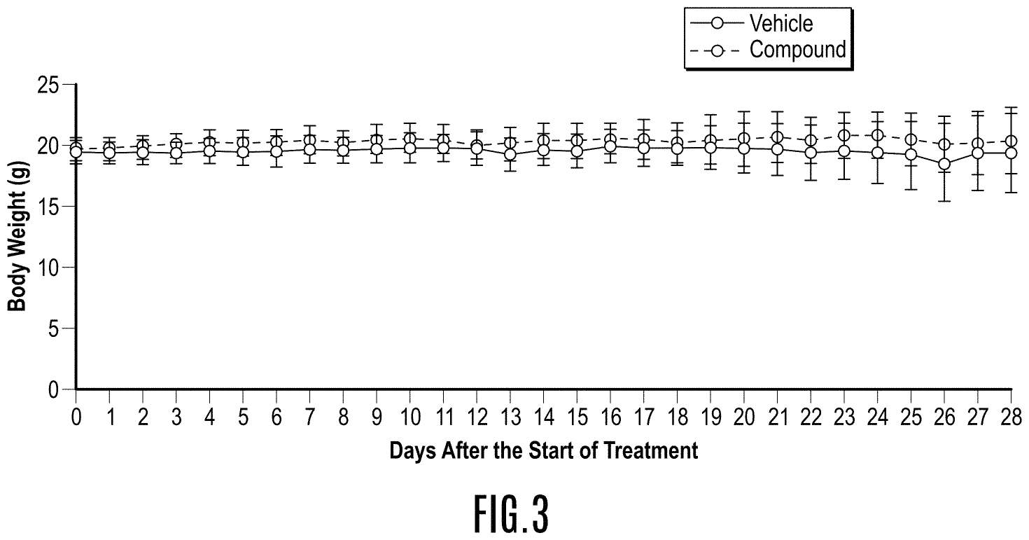

Changes in body weight are illustrated in FIG. 3. There was no significant difference in mean body weights at any day during the treatment period between the Vehicle group and the Compound group.

During the treatment period, mice found dead were as follows; one out of 8 mice was found dead in the Vehicle group.

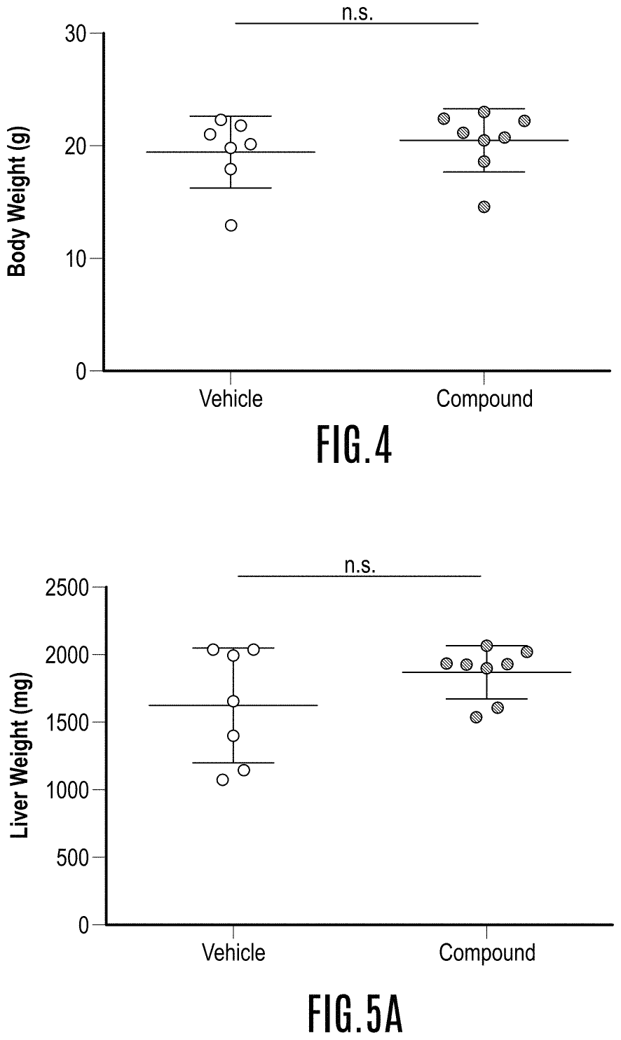

Body weight on the day of sacrifice is shown in FIG. 4 and Table 2. There was no significant difference in mean body weight on the day of sacrifice between the Vehicle group and the Compound group.

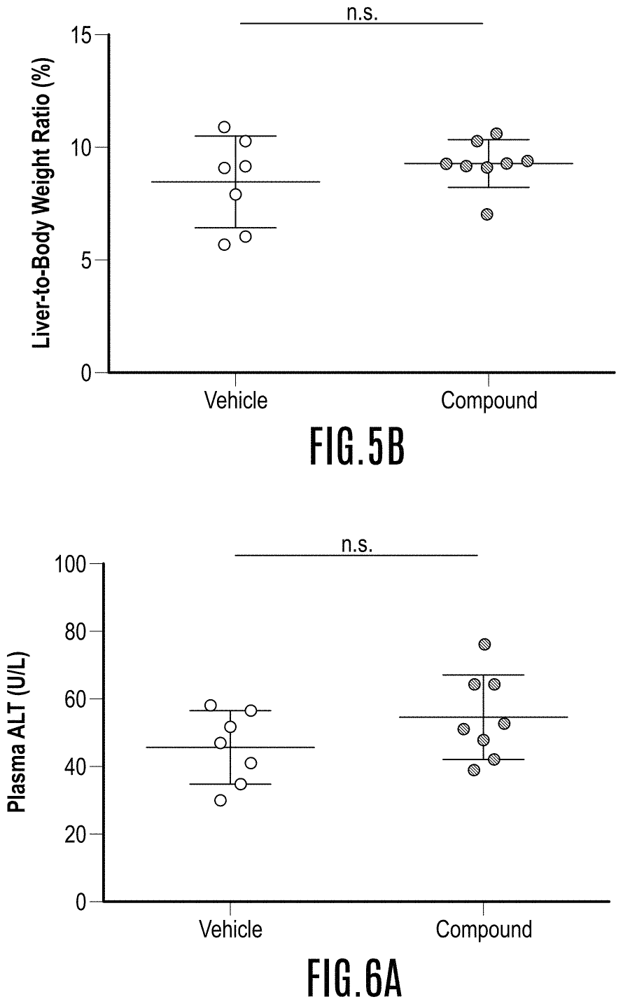

Liver weight and liver-to-body weight ratio on the day of sacrifice are shown in FIGS. 5A and 5B, respectively, and Table 2. Mean liver weight in the Compound group tended to increase compared with the Vehicle group. There was no significant difference in mean liver-to-body weight ratio between the Vehicle group and the Compound group.

TABLE 2
Body weight and liver weight

| Parameter (mean ± SD) | Vehicle (n = 7) | Compound (n = 8) |
|---|---|---|
| Body weight (g) | 19.4 ± 3.2 | 20.4 ± 2.7 |
| Liver weight (mg) | 1624 ± 422 | 1874 ± 193 |
| Liver-to-body weight ratio (%) | 8.4 ± 2.0 | 9.3 ± 1.1 |

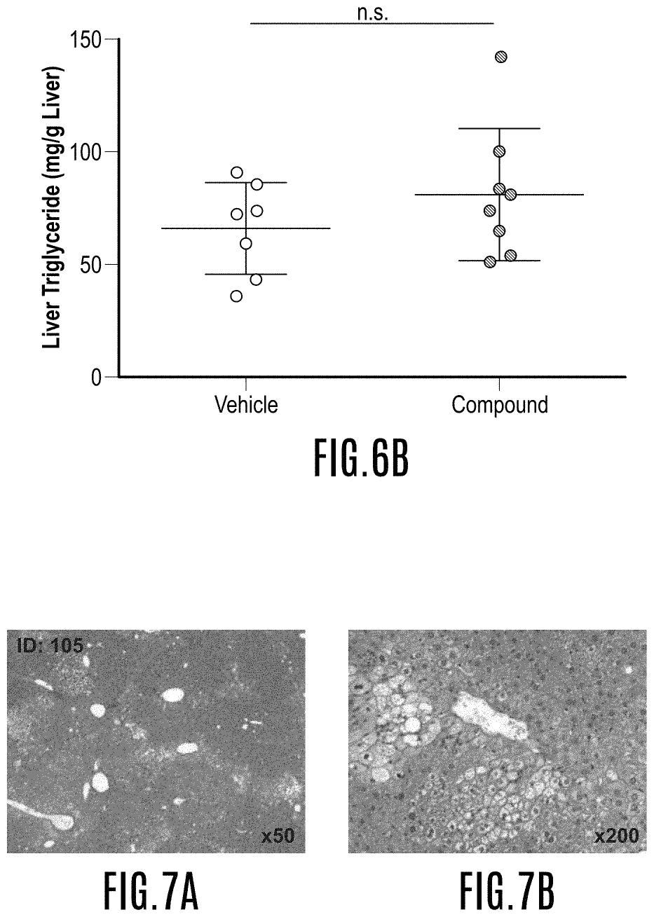

Plasma ALT is shown in FIG. 6A and Table 3. Plasma ALT level in the Compound group tended to increase compared with the Vehicle group.

Liver triglyceride is shown in FIG. 6B and Table 3. There was no significant difference in liver triglyceride contents between the Vehicle group and the Compound group.

TABLE 3
Biochemistry

| Parameter (mean ± SD) | Vehicle (n = 7) | Compound (n = 8) |
|---|---|---|
| Plasma ALT (U/L) | 46 ± 11 | 55 ± 13 |
| Liver triglyceride (mg/g liver) | 66.0 ± 20.7 | 81.3 ± 29.4 |

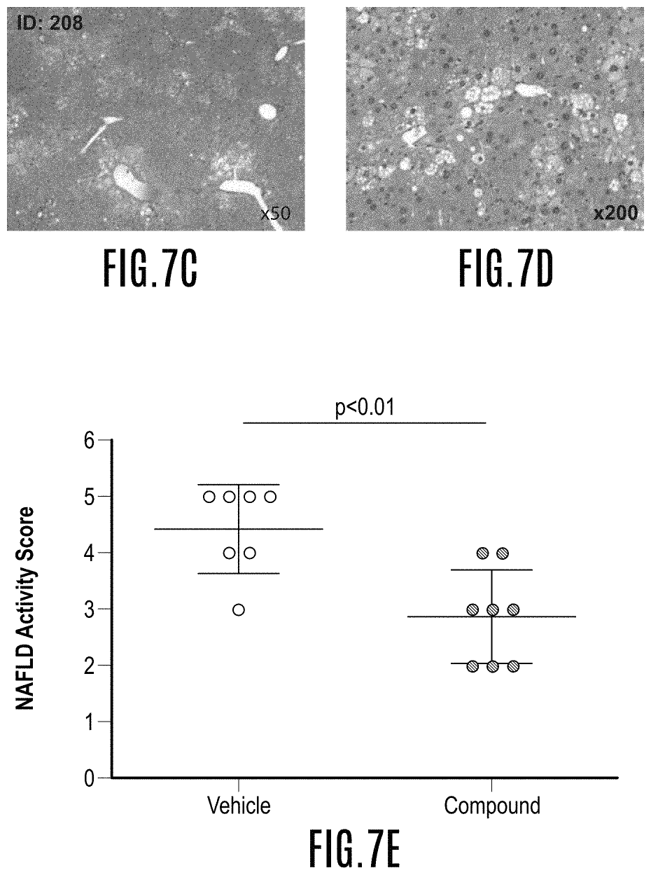

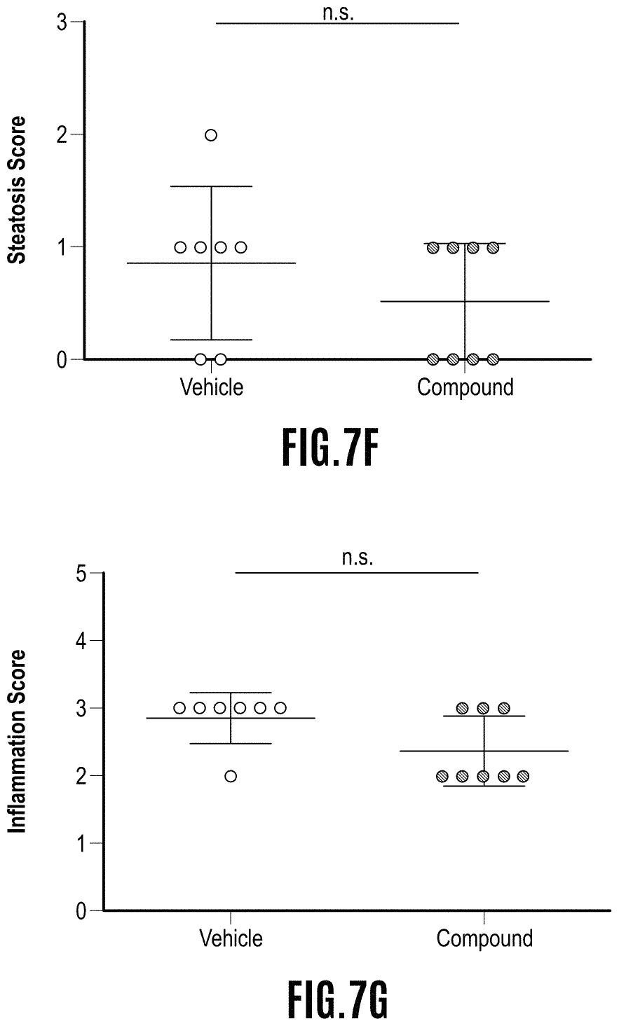

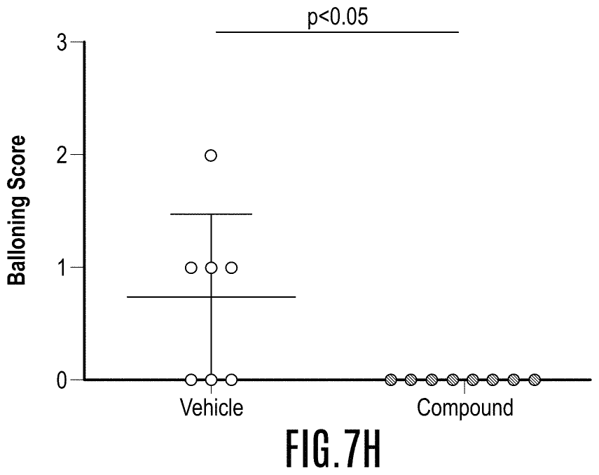

HE staining and NAFLD Activity score are illustrated in FIGS. 7(A-H) and Table 4.1.

Representative photomicrographs of HE-stained liver sections are shown in FIGS. 7(A-D).

FIG. 7E shows the NAFLD Activity Score for the murine cohort.

Further detail of the steatosis score, inflammation score, and ballooning score are provided in FIG. 7F, FIG. 7G, and FIG. 7H, respectively.

Liver sections from the Vehicle group exhibited micro- and macro-vesicular fat deposition, hepatocellular ballooning and inflammatory cell infiltration. The Compound group showed a significant decrease in NAS compared with the Vehicle group.

TABLE 4.1
NAFLD Activity score

| | | Score | | | | | | | | | | | |
|---|---|---|---|---|---|---|---|---|---|---|---|---|---|
| | | | | | | Lobular | | | | Hepatocyte | | | |
| | | Steatosis | | | | inflammation | | | | ballooning | | | NAS |
| Group | n | 0 | 1 | 2 | 3 | 0 | 1 | 2 | 3 | 0 | 1 | 2 | (mean ± SD) |
| Vehicle | 7 | 2 | 4 | 1 | — | — | — | 1 | 6 | 3 | 3 | 2 | 4.4 ± 0.8 |
| Compound | 8 | 4 | 4 | — | — | — | — | 5 | 3 | 8 | — | — | 2.9 ± 0.8 |

Components of the NAS are illustrated in Table 4.2.

TABLE 4.2
Definition of NAS Components

| Item | Score | Extent |
|---|---|---|
| | 0 | <5% |
| Steatosis | 1 | 5-33% |
| | 2 | >33-66% |
| | 3 | >66% |
| | 0 | No foci |
| Lobular | 1 | <2 foci/200x |
| Inflammation | 2 | 2-4 foci/200x |
| | 3 | >4 foci/200x |
| | 0 | None |
| Hepatocyte | 1 | Few balloon cells |
| Ballooning | 2 | Many cells/prominent ballooning |

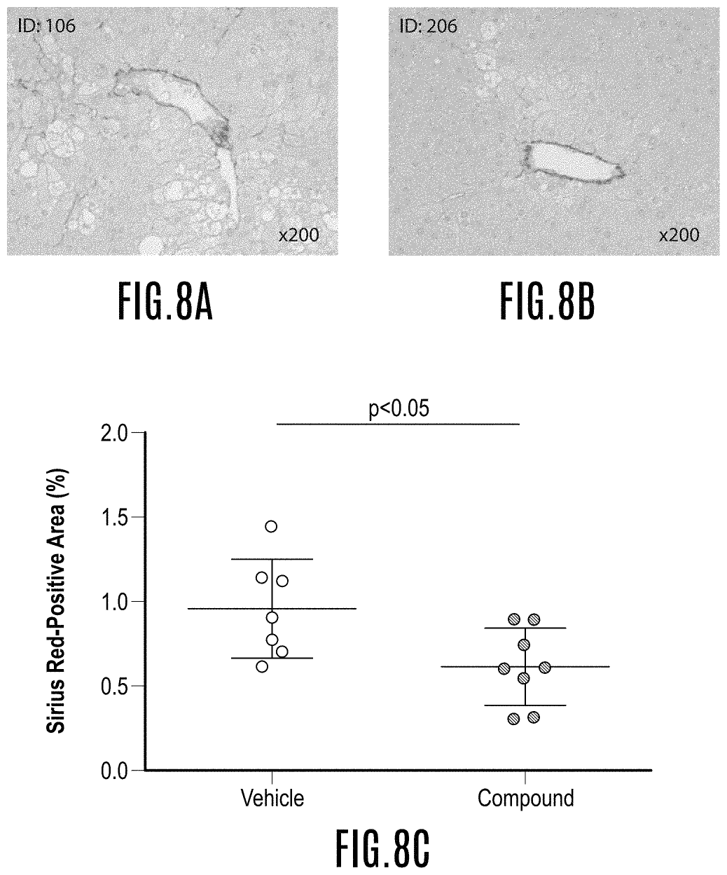

Representative photomicrographs of Sirius red-stained liver sections are shown in FIGS. 8(A-B). Liver sections from the Vehicle group showed increased collagen deposition in the pericentral region of liver lobule. The Compound group showed a significant reduction in fibrosis area (Sirius red-positive area) compared with the Vehicle group, as shown in FIG. 8C. Sirius red staining is summarized Table 5.

TABLE 5
Fibrosis area

| Parameter (mean ± SD) | Vehicle (n = 7) | Compound (n = 8) |
|---|---|---|
| Sirius red-positive area (%) | 0.96 ± 0.29 | 0.62 ± 0.23 |

### TNF-α

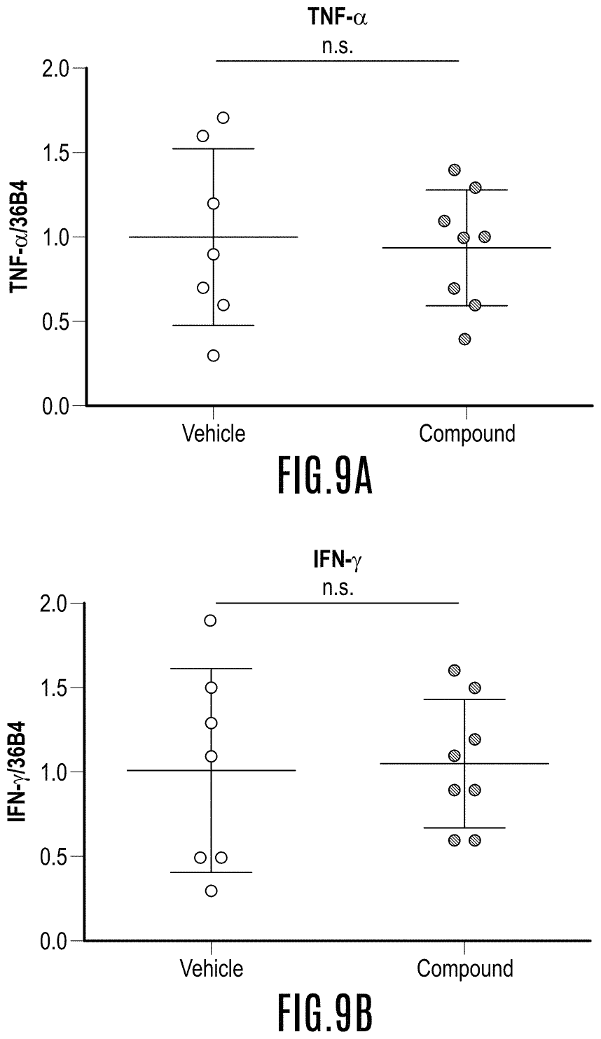

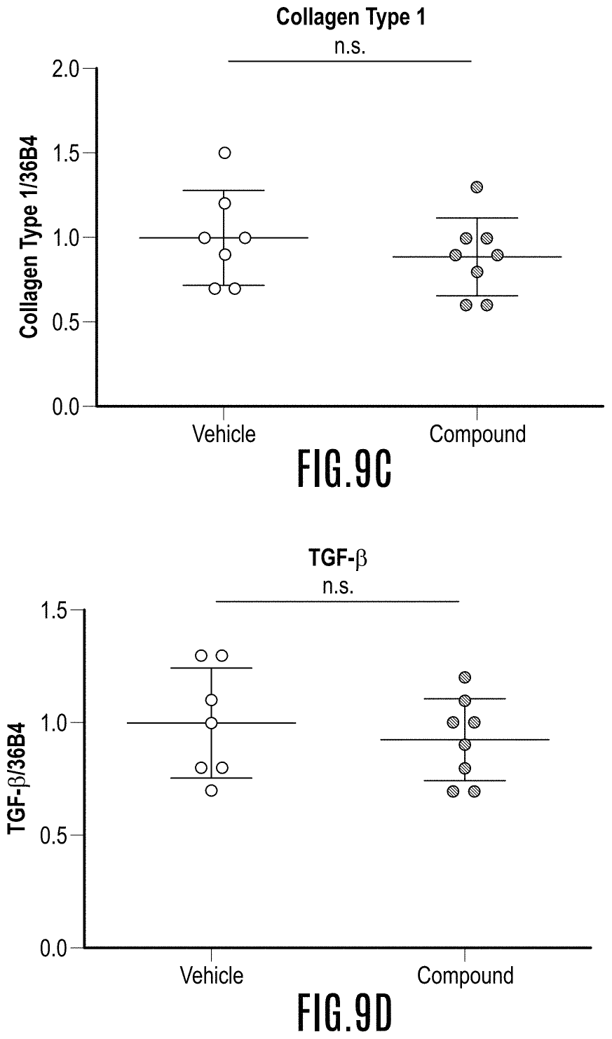

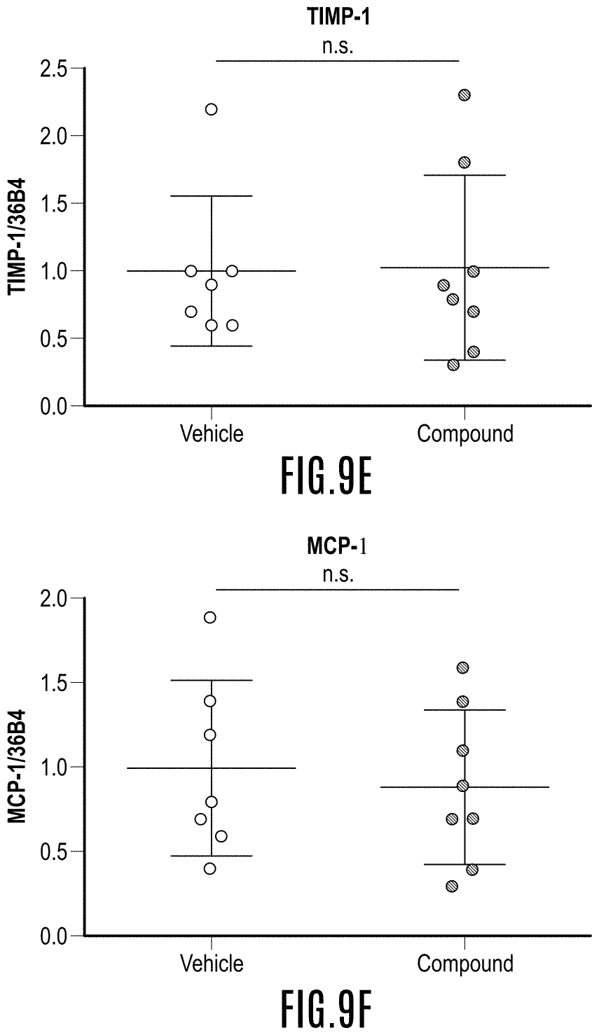

There was no significant difference in TNF-α mRNA expression level between the Vehicle group and the Compound group as illustrated in FIG. 9A and Table 6.

### INF-γ

There was no significant difference in INF-γ mRNA expression level between the Vehicle group and the Compound group as illustrated in FIG. 9B and Table 6.

### Collagen Type 1

There was no significant difference in Collagen Type 1 mRNA expression level between the Vehicle group and the Compound group as illustrated in FIG. 9C and Table 6.

### TGF-β

There was no significant difference in TGF-β mRNA expression level between the Vehicle group and the Compound group as illustrated in FIG. 9D and Table 6.

### TIMP-1

There was no significant difference in TIMP-1 mRNA expression level between the Vehicle group and the Compound group as illustrated in FIG. 9E and Table 6.

### MCP-1

There was no significant difference in MCP-1 mRNA expression level between the Vehicle group and the Compound group as illustrated in FIG. 9F and Table 6.

TABLE 6
Gene expression analyses

| Parameter (mean ± SD) | Vehicle (n = 7) | Compound (n = 8) |
|---|---|---|
| TNF-α | 1.0 ± 0.5 | 0.9 ± 0.3 |
| INF-γ | 1.0 ± 0.6 | 1.1 ± 0.4 |
| Collagen Type 1 | 1.0 ± 0.3 | 0.9 ± 0.2 |
| TGF-β | 1.0 ± 0.2 | 0.9 ± 0.2 |
| TIMP-1 | 1.0 ± 0.6 | 1.0 ± 0.7 |
| MCP-1 | 1.0 ± 0.5 | 0.9 ± 0.5 |

### Example 2: Effects of XPRO1595 in STAM Model of NASH (Continued)

In vitro additional analyses were performed to evaluate the effects of Compound in the NASH study of Example 1.

Liver and serum samples from two groups were used.

Group 1 (Vehicle): Eight NASH mice were subcutaneously administered Vehicle [normal saline] in a volume of 5 mL/kg twice weekly from 8 to 12 weeks of age.

Group 2 (Compound): Eight NASH mice were subcutaneously administered Vehicle supplemented with Compound at a dose of 10 mg/kg twice weekly from 8 to 12 weeks of age.

Serum CK-18 level was quantified by Mouse Cytokeratin 18-M30 ELISA Kit (Cusabio Biotech Co., Ltd, China).

For immunohistochemistry, sections were cut from frozen liver tissues embedded in Tissue-Tek O.C.T. compound and fixed in acetone. Endogenous peroxidase activity was blocked using 0.03% HB02 for 5 minutes, followed by incubation with Block Ace (Dainippon Sumitomo Pharma Co. Ltd., Japan) for 10 minutes.

The sections were incubated with anti-iNOS and anti-α-SMA antibody 1 hour at room temperature. After incubation with secondary antibody, enzyme-substrate reactions were performed using 3,3′-diaminobenzidine/H2O2 solution (Nichirei Bioscience Inc., Japan). The sections were incubated with anti-F4/80 antibody at room temperature for 1 hour. The sections were then incubated with biotin-conjugated secondary antibody followed by ABC reagent each for 30 minutes at room temperature. Enzyme-substrate reactions were performed using 3, 3′-diaminobenzidine/H2O2 solution (Nichirei Bioscience Inc., Japan). Profiles of primary and secondary antibodies are shown in Table 1.

For quantitative analysis of iNOS-, α-SMA- and F4/80-positive areas, bright field images of iNOS-, α-SMA- and F4/80-immunostained sections were captured around the central vein using a digital camera (DFC295; Leica, Germany) at 200-fold magnification, and the positive areas in 5 fields/section were measured using ImageJ software (National Institute of Health, USA).

TABLE 7
Profile of primary and secondary antibody
for immunochemical staining

| Test | Details of primary | Details of secondary |
|---|---|---|
| antibody | antibody | antibody |
| iNOS | Name: Rb pAb to iNOS | Name: Peroxidase labeled |
| | Manufacturer: Abcam plc. | Anti-Rabbit IgG |
| | Cat #: ab15323 | Manufacturer: Vector |
| | Dilution: 1:100 | laboratories, Inc. |
| | | Cat #: PI-1000 |
| Alpha- | Name: Rb mAb to a-SMA | Name: Peroxidase labeled |
| SMA | Manufacturer: Abcam plc. | Anti-Rabbit IgG |
| | Cat #: ab32575 | Manufacturer: Vector |
| | Dilution: 1:200 | laboratories, Inc. |
| | | Cat #: PI-1000 |
| F4/80 | Name: Anti-Mouse Rat | Name: VECTASTAIN ABC |
| | F4/80 | Rat IgG kit |
| | Manufacturer: BMA | Manufacturer: Vector |
| | Biomedicals | laboratories, Inc. |
| | Cat #: T-2006 | Cat #: PK-4004 |
| | Dilution: 1:100 | |

Statistical analyses were performed using Student's t-test on GraphPad Prism 6 (GraphPad Software Inc., USA). P values<0.05 were considered statistically significant. A trend or tendency was assumed when a one-tailed t-test returned P values<0.1. Results were expressed as mean±SD.

There was no significant difference in serum CK-18 level between the Vehicle group and the Compound group.

TABLE 8
Biochemistry (CK-18)

| Parameter (mean ± SD) | Vehicle (n = 7) | Compound (n = 8) |
|---|---|---|
| Serum CK-18 (mlU/niL) | 380.3 ± 116.7 | 403.2 ± 66.23 |

There was no significant difference in iNOS-positive area between the Vehicle group and the Compound group.

There was no significant difference in a-SMA-positive area between the Vehicle group and the Compound group.

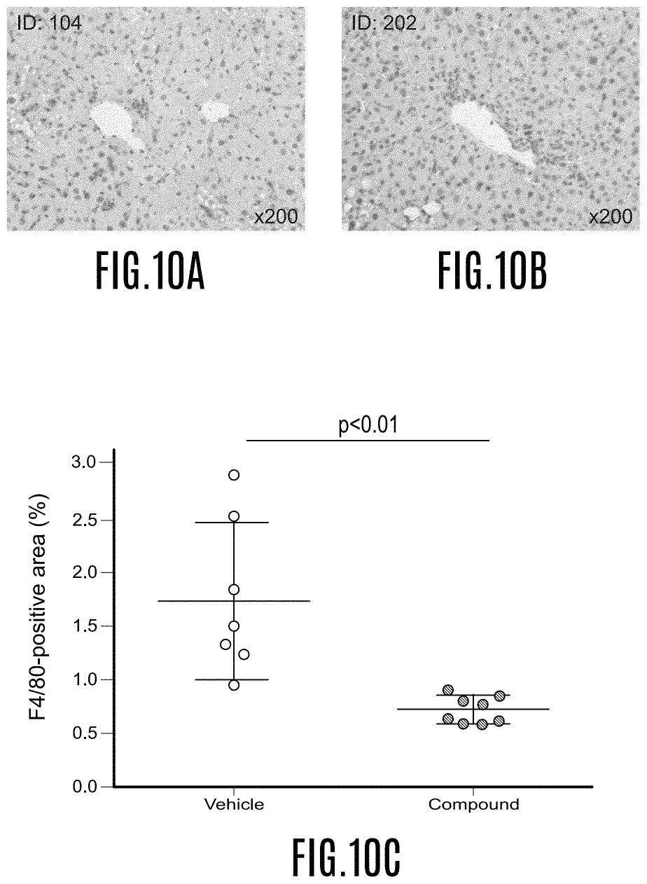

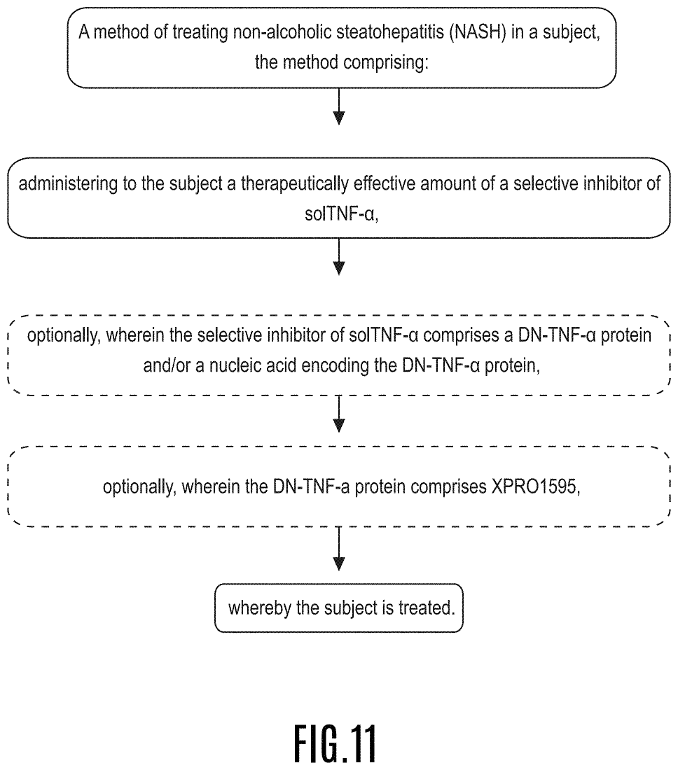

Representative photomicrographs of the F4/80-immunostained sections are shown in FIGS. 10(A-B). The Compound group showed a significant decrease in F4/80-positive area (inflammation area) compared with the Vehicle group. FIG. 10C summarizes the results of inflammation area. As shown, inflammation decreases as a result of treatment with an inhibitor of solTNF-α, specifically XPRO1595.

TABLE 3
Histological analyses (iNOS; α-SMA; F4/80)

| Parameter (mean ± SD) | Vehicle (n = 7) | Compound (n = 8) |
|---|---|---|
| iNOS-positive area (%) | 0.04 ± 0.02 | 0.04 ± 0.03 |
| F4/80-positive area (%) | 1.75 ± 0.71 | 0.73 ± 0.13 |
| Alpha-SMA-positive area (%) | 0.46 ± 0.30 | 0.47 ± 0.31 |

### Summary of Examples and Conclusion

Treatment with a selective inhibitor of solTNF-α showed significant decreases in NAS and fibrosis area compared with the Vehicle group.

NAS is one of the clinical endpoints for assessing the activity of NASH (Sanyal A J. et al., Hepatology, 2011; 54:344), and thus is the key preclinical endpoint in clinical translation. The improvement of NAS was attributable to the changes in hepatocyte ballooning, which was significantly decreased compared with the Vehicle group. Rangwala reported the close association of hepatocyte ballooning and NASH-related fibrosis (Rangwala F. et al., J. Pathol., 2011;224:401). Treatment with Compound actually significantly reduced the pathological deposition of collagen in the liver as demonstrated by Sirius red staining. Thus, reduction of hepatocyte ballooning in the group treated with the selective inhibitor of solTNF-α may underlie the anti-fibrosis effects observed in this study.

In conclusion, the selective inhibitor of solTNF-α, namely, the dominant negative TNF-α protein known as XPRO1595, showed anti-NASH and anti-fibrosis effects in this NASH model.

### INDUSTRIAL APPLICABILITY

The invention finds utility in the treatment of non-alcoholic steatohepatitis (NASH), and is therefore applicable to the medical field.

1. A method of treating non-alcoholic steatohepatitis (NASH) in a subject, the method comprising:
administering to the subject a therapeutically effective amount of a selective inhibitor of solTNF-α, whereby the subject is treated.

2. The method of claim 1, wherein the selective inhibitor of solTNF-α comprises a DN-TNF-α protein and/or a nucleic acid encoding the DN-TNF-α protein.

3. The method of claim 2, wherein the DN-TNF-α protein comprises XPRO1595.

4. The method of claim 3, wherein the method comprises administering XPRO1595 in a dose between 0.1 mg/kg and 10.0 mg/kg.

5. The method of claim 2, wherein the DN-TNF-α protein is administered: intravenously; subcutaneously; orally; via aerosol; via topical application; or via gene therapy.

6. The method of claim 5, wherein the gene therapy comprises mesenchymal stem cells expressing a construct of the DN-TNF-α protein.

7. The method of claim 2, wherein the DN-TNF-α protein is administered via gene modified autologous or allogeneic cellular therapy.

8. The method of claim 1, further comprising:
measuring in the subject at least one biomarker of NASH, wherein said biomarker of NASH is selected from the group consisting of: adiponectin (ADP), tumor necrosis factor alpha (TNF-α), leptin, c-reactive Protein (CRP), interleukin-6 (IL-6), oxidized low-density lipoprotein OxLDL), lipoprotein receptor-1 (LOX-1), interleukin-17 (IL-17), cytokeratin 18 (CK18) whole protein, cytokeratin 18 (CK18) caspase-cleaved fragments, soluble Fas (sFas), soluble Fas ligand (sFasL), ferritin, and blood neutrophil to lymphocyte (N/L) ratio; and
if the at least one biomarker measured exceeds normal threshold, then performing the step of administering to the subject said therapeutically effective amount of said selective inhibitor of solTNF-α.

9. The method of claim 1, further comprising:
measuring in the subject a NAFLD Activity Score (NAS); and
if the NAS measured is greater than or equal to 5, then performing the step of administering to the subject said therapeutically effective amount of said selective inhibitor of solTNF-α.

10. The method of claim 1, further comprising:
measuring NASH-associated fibrosis in the subject; and
if the degree of NASH-associated fibrosis measured is greater than or equal to F2, then performing the step of administering to the subject said therapeutically effective amount of said selective inhibitor of solTNF-α.

11. A selective inhibitor of solTNF-α for use in a method of treating non-alcoholic steatohepatitis, the method comprising: administering to the subject a therapeutically effective amount of the selective inhibitor of solTNF-α, optionally wherein the selective inhibitor of solTNF-α comprises a DN-TNF-α protein and/or a nucleic acid encoding the DN-TNF-α protein, optionally wherein the DN-TNF-α protein comprises a pegylated DN-TNF-α protein, and optionally wherein the DN-TNF-α protein comprises XPRO1595, whereby the subject is treated.

12. The method of claim 11, further comprising:
measuring in the subject at least one biomarker of NASH, wherein said biomarker of NASH is selected from the group consisting of: adiponectin (ADP), tumor necrosis factor alpha (TNF-α), leptin, c-reactive Protein (CRP), interleukin-6 (IL-6), oxidized low-density lipoprotein OxLDL), lipoprotein receptor-1 (LOX-1), interleukin-17 (IL-17), cytokeratin 18 (CK18) whole protein, cytokeratin 18 (CK18) caspase-cleaved fragments, soluble Fas (sFas), soluble Fas ligand (sFasL), ferritin, and blood neutrophil to lymphocyte (N/L) ratio; and
if the at least one biomarker measured exceeds normal threshold, then performing the step of administering to the subject said therapeutically effective amount of said selective inhibitor of solTNF-α.

13. The method of claim 11, further comprising:
measuring in the subject a NAFLD Activity Score (NAS); and
if the NAS measured is greater than or equal to 5, then performing the step of administering to the subject said therapeutically effective amount of said selective inhibitor of solTNF-α.

14. The method of claim 11, further comprising:
measuring NASH-associated fibrosis in the subject; and
if the degree of NASH-associated fibrosis measured is greater than or equal to F2, then performing the step of administering to the subject said therapeutically effective amount of said selective inhibitor of solTNF-α.

15. The method of claim 11, wherein said XPRO1595 is administered: intravenously; subcutaneously; orally; via aerosol; via topical application; or via gene therapy.

16. The method of claim 11, wherein said XPRO1595 is administered in a dose between 0.1 mg/kg and 10.0 mg/kg.


SEQUENCE LISTING (3 sequences)

SEQ ID NO: 1 - DNA, 495 bp, Homo sapiens
atgcaccaccaccaccaccacgtacgctcctcctcccgcactccgtccgacaaaccggtagctcacgtagtagctaacccgcaggctgaaggtcagctgcagtggctgaaccgccgcgctaacgctctgctggctaacggtgtagaactgcgcgacaaccagctggtagtaccgtccgaaggtctgtacctgatctactcccaggtactgttcaaaggtcagggttgtccgtccactcacgtactgctgactcacactatctcccgcatcgctgtatcctaccagactaaagtaaacctgctgtccgctatcaaatccccgtgtcagcgcgaaactccggaaggtgctgaagctaaaccgtggtacgaaccgatctacctgggtggtgtattccagctggaaaaaggtgaccgcctgtccgctgaaatcaaccgcccggactacctggacttcgctgaatccggtcaggtatacttcggtatcatcgctctgtga

SEQ ID NO: 2 - protein, 164 aa, Homo sapiens
MHHHHHHVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 157 aa, Homo sapiens
VRSSSRTPSDKPVAHVVANPQAEGQLQWLNRRANALLANGVELRDNQLVVPSEGLYCIYSQVLFKGQGCPSTHVLLTHTISRIAVSYQTKVNLLSAIKSPCQRETPEGAEAKPWYEPITLGGVFQLEKGDRLSAEINRPDYLDFAESGQVYFGIIAL